(19) United States
(12) Patent Application Publication
(10) Pub. No.: US 2005/0054614 A1
(43) Pub. Date: Mar. 10, 2005

(54) Methods of inhibiting leukocyte accumulation

(76) Inventors: Thomas G. Diacovo, St. Louis, MO (US); Joel S. Hayflick, Seattle, WA (US); Kamal D. Puri, Lynnwood, WA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/918,825

(22) Filed: Aug. 13, 2004

Related U.S. Application Data
(60) Provisional application No. 60/495,370, filed on Aug. 14, 2003.
(60) Provisional application No. 60/540,036, filed on Jan. 28, 2004.

(51) Int. Cl. A61K 31/675; A61K 31/445

(52) U.S. Cl. 514/80; 514/317

(57) ABSTRACT

The invention relates generally to phosphoinositide 3-kinases (PI3Ks), and more particularly to methods of inhibiting leukocyte accumulation comprising selectively inhibiting phosphoinositide 3-kinase delta (PI3Kδ) activity in endothelial cells.

### CROSS-REFERENCE TO RELATED APPLICATIONS

The benefit under 35 U.S.C. §119(e) of U.S. provisional patent application Ser. Nos. 60/495,370 filed Aug. 14, 2003, and 60/540,036 filed Jan. 28, 2004, the entire disclosures of which are incorporated herein by reference, is claimed.

### GOVERNMENT RIGHTS IN THE INVENTION

Scientific work relating to the present invention was supported in part by the United States government under grant no. RO1 HL63244-O1A1 awarded by the National Heart, Lung, and Blood Institute. The United States government may have certain rights in this invention.

### FIELD OF THE INVENTION

The invention relates generally to phosphoinositide 3-kinases (PI3Ks), and more particularly to methods of inhibiting leukocyte accumulation, comprising selectively inhibiting phosphoinositide 3-kinase delta (PI3Kδ) activity in endothelial cells.

### BACKGROUND OF THE INVENTION

Inflammatory responses may result from infection with pathogenic organisms and viruses, noninfectious means such as trauma or reperfusion following myocardial infarction or stroke, immune responses to foreign antigens, and autoimmune diseases. Inflammatory responses are notably associated with the influx of leukocytes and/or leukocyte chemotaxis.

The recruitment of leukocytes into inflamed tissues is dependent upon a series of adhesive events that occur between these cells and the endothelial cells of the microvasculature [Springer, Cell 76:301-314 (1994); and, Butcher et al., Science 272:60-66 (1996)]. Tissue injury initiates this adhesion process by locally releasing mediators of inflammation including but not limited to histamine, TNFα and IL-1 that rapidly convert the endothelial cell surface to a proadhesive state. The conversion of the endothelial cell surface to a proadhesive state includes the upregulation of P-selectin and E-selectin on the luminal surface of blood vessels. P-selectin and E-selectin subsequently interact with constitutively-expressed carbohydrate ligands on circulating leukocytes to promote rapid attachment and rolling of these cells in preparation for transendothelial migration.

Selectin-mediated adhesion is critical to transendothelial migration as it facilitates the engagement of secondary leukocyte adhesion receptors including but not limited to the β₂-integrins with intracellular adhesion molecules (ICAMs) expressed on the surface of inflamed vascular endothelium. Selectin-mediated adhesion requires leukocyte stimulation by locally-produced chemoattractants including but not limited to IL-8 and LTB₄, and subsequently results in integrin-mediated stabilization of interactions between these cells and the vasculature endothelial cells. Leukocytes eventually transmigrate across the endothelial cell barrier towards inflammatory foci in response to a bacterial and/or host-derived chemoattractant(s) [Luster, N. Engl. J. Med. 338:436-445 (1998)]. Failure to complete any of these steps will impede leukocyte accumulation in inflamed tissue, as evidenced by leukocyte adhesion deficiency syndromes I and II [Kishimoto et al., Cell, 50:193-202 (1987); and, Etzioni, Pediatr. Res., 39:191-198 (1996)].

Class I phosphoinositide 3-kinases (PI 3-kinases; PI3Ks) are known to play a pivotal role in the ability of leukocytes to undergo chemotaxis as the lipid products they generate, including but not limited to phosphatidylinositol (3,4,5)-trisphosphate (PIP3), are critical for promoting asymmetric F-actin synthesis, and thus leukocyte cell polarization [Wymann et al., Immunol. Today. 21:260-264 (2000); Fruman et al., Semin. Immunol. 14:7-18 (2002); Rickert et al., Trends Cell Biol., 10:466473 (2000); and, Weiner et al., Nat. Cell Biol., 1:75-81 (1999)]. The function of class I PI3Ks, however, is not limited to directed migration, in that they are also required for phagocytosis and generation of oxygen radicals in response to chemoattractants including but not limited to fMLP [Arcaro et al., Biochem. J., 298:517-520 (1994); Cadwallader et al., J. Immunol., 169:3336-3344 (2002); Sasaki et al., Science, 287:1040-1046 (2000); Ninomiya et al., J. Biol. Chem., 269:22732-22737 (1994); Bharadwaj et al., J. Immunol. 166:6735-6741 (2001))]. The ability of class I PI3Ks to regulate these processes in leukocytes relies on PIP₃ mediated recruitment of two lipid-binding protein kinases, phosphatidylinositol-dependent kinase 1 (PDK1) and protein kinase B/Akt, both of which can interact with this PI-derivative via their pleckstrin homology domains. Association of these kinases with PIP₃ at the plasma membrane brings them into close proximity, facilitating the phosphorylation and activation of Akt by PDK1 [Cantley, Science, 296:1655-1657 (2002)]. These proteins are, in turn, responsible for many of the downstream signaling events associated with PI3K activity.

Structurally, class I PI3Ks exist as heterodimeric complexes, consisting of a p110 catalytic subunit and a p55, p85, or p101 regulatory subunit. There are four p110 catalytic-subunits, which are classified as p110α, p110β, p110γ, and p110δ [Wymann et al., Biochim. Biophys. Acta., 1436:127-150 (1998); and, Vanhaesebroeck et al., Trends Biochem. Sci., 22:267-272 (1997)]. Class I PI3Ks can be further divided into two subclasses (Ia and Ib) based on their mechanism of activation. The class Ia subgroup contains p110α, p110α, and p110δ, each of which associates with the p85 regulatory protein and is activated by receptor tyrosine kinases [Wymann et al., Biochim. Biophys. Acta., 1436:127-150 (1998); Curnock et al., Immunology, 105:125-136 (2002); and, Stein et al., Mol. Med. Today, 6:347-357 (2000)]. By contrast, the class Ib subgroup consists solely of p110γy, which associates with the p101 regulatory subunit, and is stimulated by G protein βγ subunits in response to chemoattractants. Neutrophils express all four members of class I PI3Ks.

Evidence supporting the class I PI3Ks involvement in neutrophil cell migration is found in the ability of non-selective class I PI3K inhibitors, such as LY294002 and wortmannin, to mitigate neutrophil chemotaxis. Moreover, chemoattractant-directed migration of neutrophils has been reduced in mice deficient for p110γ catalytic subunit expression [Sasaki et al., Science, 287:1040-1046 (2000); Knall et al., Proc. Natl. Acad. Sci. U.S.A., 94:3052-3057 (1997); Hannigan et al., Proc. Natl. Acad. Sci. U.S.A., 99:3603-3608 (2002); and, Hirsch et al., Science, 287:1049-1053 (2000)]. The phosphoinositide 3-kinase (PI3K) catalytic subunit p110δ is thought to play a role at sites of inflammation by contributing solely to chemoattractant-directed neutrophil migration.

PI3K inhibitors that are selective for PI3Kδ have been disclosed in U.S. Patent Publication 2002/161014 A1. Recently, the effects of a class I small molecule inhibitor specific for the PI3Kδ catalytic subunit have been studied [Sadhu et al., J. Immunol., 170:2647-2654 (2003)]. This small molecule inhibitor was shown to block up to 65% of fMLP-induced PIP3 generation in neutrophils as well as directed-migration of these cells on surface-immobilized ICAM-1 in response to this microbial product. Thus, Sadhu et al. demonstrated that the lipid kinase activity of PI3Kδ is required for neutrophil directional migration to fMLP (using an under-agarose assay system). PI3Kδ inhibition affected both the number of neutrophils that were able to migrate towards this bacterial product and the distance they were able to migrate.

Leukocyte accumulation in inflamed tissues relies on their ability to form adhesive interactions with inflamed vascular endothelium in response to chemoattractant-guided migration. Previously, it was known that the phosphoinositide 3-kinase (PI3K) catalytic subunit p110δ is expressed in neutrophils. In fact, previous reports suggest that p110δ expression is largely restricted to leukocytes. The prior art, thus, merely suggests that p110δ plays a role in neutrophil accumulation at sites of inflammation by contributing solely to chemoattractant-directed migration, and a role for class I PI3Ks in modulating the ability of cytokine-stimulated vascular endothelium to promote adhesive interactions with neutrophils has not been previously demonstrated.

### SUMMARY OF THE INVENTION

The invention provides methods which inhibit leukocyte accumulation.

According to one embodiment of the invention, a method of inhibiting leukocyte accumulation comprises selectively inhibiting phosphoinositide 3-kinase delta (PI3Kδ) activity in endothelial cells. In one aspect of this embodiment, the method comprises administering an amount of a phosphoinositide 3-kinase delta (PI3Kδ) selective inhibitor effective to inhibit p110 delta (p110δ) in endothelial cells.

According to another embodiment, a method of inhibiting leukocyte tethering to endothelial cells comprises selectively inhibiting phosphoinositide 3-kinase delta (PI3Kδ) activity in endothelial cells. In one aspect of this embodiment, the method comprises administering an amount of a PI3Kδ selective inhibitor effective to inhibit leukocyte tethering to endothelial cells.

According to an additional embodiment, a method of inhibiting leukocyte transmigration comprises selectively inhibiting phosphoinositide 3-kinase delta (PI3Kδ) activity in endothelial cells. In one aspect of this embodiment, the method comprises administering an amount of a PI3Kδ selective inhibitor effective to inhibit leukocyte transmigration into inflamed tissue.

### DETAILED DESCRIPTION

The disclosed methods may be used to treat individuals having an inflammatory condition where leukocytes are found to be accumulating at the site of insult or inflamed tissue. An individual, however, need not be afflicted by an inflammatory condition in order for treatment in accordance with the methods of the invention to be warranted, i.e., the methods may be used to prophylactically, i.e., to prevent onset and/or recurrence of inflammatory conditions.

Certain inflammatory conditions of the lungs including but not limited to chronic obstructive pulmonary disease and acute respiratory distress syndrome are often associated with sustained neutrophil accumulation. Sustained neutrophil accumulation can result in undesired side effects including but not limited to the destruction of normal tissue architecture [Dallegri et al., Inflamm. Res., 46:382-391 (1997)]. Because the methods of the invention inhibit undesirable leukocyte accumulation, subsequent tissue damage caused by production and release of mediators from the leukocytes that cause oxygen free radical- and protease-mediated tissue damage can be attenuated or eliminated. Importantly, inhibition of PI3Kδ function does not appear to effect biological functions including but not limited to viability and fertility. Thus, PI3Kδ is an attractive target for the development of drugs that may be of benefit in the treatment of inflammatory conditions.

“Inflammatory condition” as used herein refers to a condition characterized by redness, heat, swelling and pain (i.e., inflammation) that typically involves tissue injury or destruction. Inflammatory conditions are notably associated with the influx of leukocytes and/or leukocyte chemotaxis. Inflammatory conditions may result from infection with pathogenic organisms or viruses and from noninfectious events including but not limited to trauma or reperfusion following myocardial infarction or stroke, immune responses to foreign antigens, and autoimmune responses. Accordingly, inflammatory conditions amenable to treatment with the methods and compounds of the invention encompass conditions associated with reactions of the specific defense system, conditions associated with reactions of the non-specific defense system, and conditions associated with inflammatory cell activation.

As used herein, the term “specific defense system” refers to the component of the immune system that reacts to the presence of specific antigens. Examples of inflammatory conditions resulting from a response of the specific defense system include but are not limited to the classical response to foreign antigens, autoimmune diseases, and delayed type hypersensitivity response mediated by B-cells and/or T-cells (i.e., B-lymphocytes and/or T-lymphocytes). Chronic inflammatory diseases, the rejection of solid transplanted tissue and organs including but not limited to kidney and bone marrow transplants, and graft versus host disease (GVHD), are further examples of inflammatory conditions resulting from a response of the specific defense system.

The term “non-specific defense system” as used herein refers to inflammatory conditions that are mediated by leukocytes that are incapable of immunological memory (e.g., granulocytes including but not limited to neutrophils, eosinophils, and basophils, mast cells, monocytes, macrophages). Examples of inflammatory conditions that result, at least in part, from a reaction of the non-specific defense system include but are not limited to adult (acute) respiratory distress syndrome (ARDS), multiple organ injury syndromes, reperfusion injury, acute glomerulonephritis, reactive arthritis, dermatitis with acute inflammatory components, acute purulent meningitis, other central nervous system inflammatory conditions including but not limited to stroke, thermal injury, inflammatory bowel disease, granulocyte transfusion associated syndromes, and cytokine-induced toxicity.

The therapeutic methods of the invention include methods for the amelioration of conditions associated with inflammatory cell activation. “Inflammatory cell activation” refers to the induction by a stimulus (including but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including but not limited to, major histocompatability antigens or cell adhesion molecules) in inflammatory cells (including but not limited to monocytes, macrophages, T lymphocytes, B lymphocytes, granulocytes (polymorphonuclear leukocytes including neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory condition.

“Autoimmune disease” as used herein refers to any group of inflammatory conditions in which tissue injury is associated with humoral or cell-mediated responses to the body's own constituents. “Allergic disease” as used herein refers to any symptoms, tissue damage, or loss of tissue function resulting from allergy. “Arthritic disease” as used herein refers to any inflammatory condition that is characterized by inflammatory lesions of the joints attributable to a variety of etiologies. “Dermatitis” as used herein refers to any of a large family of inflammatory conditions of the skin that are characterized by inflammation of the skin attributable to a variety of etiologies. “Transplant rejection” as used herein refers to any immune reaction directed against grafted tissue (including but not limited to organs or cells (e.g., bone marrow) that is characterized by a loss of function of the grafted and surrounding tissues, pain, swelling, leukocytosis, and/or thrombocytopenia.

The invention provides methods of inhibiting leukocyte accumulation comprising selectively inhibiting phosphoinositide 3-kinase delta (PI3Kδ) activity in endothelial cells. Thus, the methods of the invention include inhibiting leukocyte accumulation by inhibiting an upstream target in the pathway that selectively activates PI3Kδ in endothelial cells. In one aspect of this embodiment, the method comprises administering an amount of a phosphoinositide 3-kinase delta (PI3Kδ) selective inhibitor effective to inhibit p110 delta (p110δ) in endothelial cells.

As used herein, the term “selectively inhibiting phosphoinositide 3-kinase delta (PI3Kδ) activity” generally refers to inhibiting the activity of the PI3Kδ isozyme more effectively than other isozymes of the PI3K family. Similarly, the term “PI3Kδ selective inhibitor” generally refers to a compound that inhibits the activity of the PI3Kδ isozyme more effectively than other isozymes of the PI3K family. A PI3Kδ selective inhibitor compound is therefore more selective for PI3Kδ than conventional PI3K inhibitors such as wortmannin and LY294002, which are “nonselective PI3K inhibitors.”

As used herein, the term “amount effective” means a dosage sufficient to produce a desired or stated effect.

In another embodiment, the invention provides methods of inhibiting leukocyte tethering to endothelial cells comprises selectively inhibiting phosphoinositide 3-kinase delta (PI3Kδ) activity in endothelial cells. In one aspect of this embodiment, the method comprises administering an amount of a PI3Kδ selective inhibitor effective to inhibit leukocyte tethering to endothelial cells.

In a further embodiment, the invention provides methods of inhibiting leukocyte transmigration comprises selectively inhibiting phosphoinositide 3-kinase delta (PI3Kδ) activity in endothelial cells. In one aspect of this embodiment, the method comprises administering an amount of a PI3Kδ selective inhibitor effective to inhibit leukocyte transmigration into inflamed tissue.

The disclosed methods may affect inflammatory conditions mediated by one or more components of the PI3K/Akt signal transduction pathway of endothelial cells. Therefore, the methods may inhibit or reduce AKT-activity of endothelial cells, e.g., as measured by AKT-phosphorylation. Additionally, the disclosed methods may inhibit or reduce PDK1 enzyme activity of endothelial cells.

In one embodiment of the invention, inhibition of p110δ in leukocytes does not affect leukocyte accumulation and/or leukocyte tethering to endothelial cells. The disclosed methods may affect inflammatory conditions without substantially inhibiting one or more components of the p38 mitogen-activated kinase (p38 MAPK) pathway in endothelial cells and/or leukocytes. The disclosed methods also may not substantially inhibit the following pathways in endothelial cells and/or leukocytes: Rac GTPase, and phosphodiesterases, specifically PDE4.

In the methods of the invention, the leukocytes are selected from the group consisting of neutrophils, eosinophils, basophils, T-lymphocytes, B-lymphocytes, monocytes, macrophages, dendritic cells, Langerhans cells, and mast cells. In one aspect, the leukocytes are neutrophils.

Leukocyte accumulation involves leukocyte adhesion to endothelial cells and then transmigration of the leukocytes through an endothelial cell layer. Leukocyte adhesion to endothelial cells is a labile process including initial leukocyte tethering, followed by leukocyte rolling along the vessel wall, and firm adhesion to the wall. Adhesion is typically initiated in response to extravascular inflammation mediators or stimuli, which cause the leukocytes and/or endothelial cells to become adhesive. Thus, leukocyte adhesion to endothelial cells is typically initiated in response to an inflammation mediator. Inflammation mediators, which cause the leukocytes and/or endothelial cells to become adhesive include but are not limited to histamine, tumor necrosis factor alpha (TNF-alpha), interleukin 1 alpha (IL-1 alpha), interleukin 1 beta (IL-1 beta), Duffy antigen/receptor for chemokines (DARC), lymphotactin, stromal cell-derived factor-1 (SDF-1), transforming growth factor beta (TGF-beta), gamma-interferon (IFN-gamma), leukotriene B4 (LTB4), thrombin, formyl-methionyl-leucyl-phenylalanine (fMLP), lipopolysaccharides (LPS), platelet-activating factor (PAF), and lysophospholipids.

The adhesivity induced in these cells can result in temporary adhesion between the leukocytes and the endothelial cells, typically referred to as leukocyte tethering. Leukocyte tethering is generally mediated by interactions between selectin receptors including but not limited to E-selectin and P-selectin on endothelial cells and corresponding ligands present on leukocytes. The corresponding ligands are generally sialylated, fucosylated glycoconjugates. In some cases, selectin receptors including but not limited to L-selectin are present on leukocytes and the corresponding ligands are present on endothelial cells. In one embodiment of the invention, the methods of the invention inhibit interactions between E-selectin and/or P-selectin on endothelial cells and the corresponding ligands on leukocytes.

The leukocyte tethering and shear forces due to blood flow can result in leukocytes rolling along a vessel wall. As in the case of leukocyte tethering, leukocyte rolling is generally mediated by interactions between selectin receptors and corresponding ligands. Typically, the methods of the invention modulate selectin-mediated leukocyte adhesion to endothelial cells, and thus affect leukocyte tethering and leukocyte rolling. Further, the methods of the invention can increase a mean rolling velocity of leukocytes along the endothelial cell surfaces. According to one aspect, the mean leukocyte rolling velocity is increased by at least about 200 percent. In an additional aspect, the mean rolling velocity is increased by at least about 400 percent, and in yet a further aspect by at least about 800 percent.

Upon further pro-inflammatory stimulation (typically with activating chemoattractants and/or chemokines), some leukocytes stick or firmly adhere to the endothelial cells, resulting in firm adhesion resistant to shear forces within the blood vessel. Endogenous cytokines and chemoattractants including but not limited to TNFα and LTB₄ are essential for promoting both leukocyte attachment to inflamed microvessels as well as directed migration of these cells [Xing et al., Am. J. Pathol., 143:1009-1015 (1993); and, Yamasawa et al., Inflammation, 23:263-274 (1999)]. Firm adhesion is generally mediated by interactions between integrin receptors including but not limited to LFA-1, Mac-1, α₄β₇, and VLA-4 on the leukocytes and immunoglobin superfamily (IgSF) ligands including but not limited ICAM-1, PECAM-1, MAd-CAM-1, and VCAM-1 on the endothelial cells. In one embodiment, the methods of the invention do not substantially inhibit integrin-mediated firm adhesion of leukocytes to endothelial cells.

Ultimately, the firmly adhered leukocytes transmigrate between endothelial cells into inflamed tissues, typically in response to chemoattractants. According to one embodiment, the methods of the invention inhibit leukocyte transmigration into inflamed tissue. In one aspect of this embodiment, the methods inhibit transmigration into inflamed tissue by at least about twenty percent, in another aspect by at least about twenty five percent, and in a further aspect by at least about thirty percent. The inflamed tissue may generally be any tissue. According to one aspect of the invention, the inflamed tissue is pulmonary tissue.

Autoimmune conditions which may be treated using an inhibitor of the invention include but are not limited to connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. The inhibitors of the invention may also be useful in the treatment of allergic reactions and conditions including but not limited to anaphylaxis, serum sickness, drug reactions, food allergies, insect venom allergies, mastocytosis, allergic rhinitis, hypersensitivity pneumonitis, urticana, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, contact allergies including but not limited to asthma (particularly, allergic asthma), and other respiratory problems.

Thus, in various embodiments, the invention provides methods of treating various inflammatory conditions including but not limited to arthritic diseases such as rheumatoid arthritis (RA), osteoarthritis, gouty arthritis, spondylitis, and reactive arthritis; Behcet's syndrome; sepsis; septic shock; endotoxic shock; gram negative sepsis; gram positive sepsis; toxic shock syndrome; multiple organ injury syndrome secondary to septicemia, trauma, or hemorrhage; ophthalmic disorders including but not limited to allergic conjunctivitis, vernal conjunctivitis, uveitis, and thyroid-associated ophthalmopathy; eosinophilic granuloma; pulmonary or respiratory conditions including but not limited to asthma, chronic bronchitis, allergic rhinitis, adult respiratory distress syndrome (ARDS), severe acute respiratory syndrome (SARS), chronic pulmonary inflammatory diseases (e.g., chronic obstructive pulmonary disease), silicosis, pulmonary sarcoidosis, pleurisy, alveolitis, vasculitis, pneumonia, bronchiectasis, hereditary emphysema, and pulmonary oxygen toxicity; ischemic-reperfusion injury, e.g., of the myocardium, brain, or extremities; fibrosis including but not limited to cystic fibrosis; keloid formation or scar tissue formation; atherosclerosis; autoimmune diseases including but not limited to systemic lupus erythematosus (SLE), lupus nephritis, autoimmune thyroiditis, multiple sclerosis, some forms of diabetes, and Reynaud's syndrome; tissue or organ transplant rejection disorders including but not limited to graft versus host disease (GVHD) and allograft rejection; chronic or acute glomerulonephritis; inflammatory bowel diseases including but not limited to Crohn's disease, ulcerative colitis and necrotizing enterocolitis; inflammatory dermatitis including but not limited to contact dermatitis, atopic dermatitis, psoriasis, and urticaria; fever and myalgias due to infection; central or peripheral nervous system inflammatory conditions including but not limited to meningitis (e.g., acute purulent meningitis), encephalitis, and brain or spinal cord injury due to minor trauma; Sjorgren's syndrome; diseases involving leukocyte diapedesis; alcoholic hepatitis; bacterial pneumonia; community acquired pneumonia (CAP); neumocystis carinii pneumonia (PCP); antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; cytokine-induced toxicity; stroke; pancreatitis; myocardial infarction, respiratory syncytial virus (RSV) infection; and spinal cord injury.

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The ability of the PI3Kδ selective inhibitors of the invention to treat arthritis can be demonstrated in a murine collagen-induced arthritis model [Kakimoto et al., Cell. Immunol., 142:326-337 (1992)], in a rat collagen-induced arthritis model [Knoerzer et al., Toxicol. Pathol., 25:13-19 (1997)], in a rat adjuvant arthritis model [Halloran et al., Arthritis Rheum., 39:810-819 (1996)], in a rat streptococcal cell wall-induced arthritis model [Schimmer et al., J. Immunol., 160:1466-1477 (1998)], or in a SCID-mouse human rheumatoid arthritis model [Oppenheimer-Marks et al., J. Clin. Invest., 101:1261-1272(1998)]. The ability of the PI3Kδ selective inhibitors to treat Lyme arthritis can be demonstrated according to the method of Gross et al., Science, 218:703-706, (1998).

The ability of the PI3Kδ selective inhibitors to treat asthma can be demonstrated in a murine allergic asthma model according to the method of Wegner et al., Science, 247:456-459 (1990), or in a murine non-allergic asthma model according to the method of Bloemen et al., Am. J. Respir. Crit. Care Med. 153:521-529 (1996).

The ability of the PI3Kδ selective inhibitors to treat inflammatory lung injury can be demonstrated in a murine oxygen-induced lung injury model according to the method of Wegner et al., Lung, 170:267-279 (1992), in a murine immune complex-induced lung injury model according to the method of Mulligan et al., J. Immunol., 154:1350-1363 (1995), or in a murine acid-induced lung injury model according to the method of Nagase et al., Am. .J. Respir. Crit. Care Med., 154:504-510 (1996).

The ability of the PI3Kδ selective inhibitors to treat inflammatory bowel disease can be demonstrated in a murine chemical-induced colitis model according to the method of Bennett et al., J. Pharmacol., Exp. Ther., 280:988-1000 (1997).

The ability of the PI3Kδ selective inhibitors to treat autoimmune diabetes can be demonstrated in an NOD mouse model according to the method of Hasagawa et al., Int. Immunol. 6:831-838 (1994), or in a murine streptozotocin-induced diabetes model according to the method of Herrold et al., Cell Immunol. 157:489-500 (1994).

The ability of the PI3Kδ selective inhibitors to treat inflammatory liver injury can be demonstrated in a murine liver injury model according to the method of Tanaka et al., J. Immunol., 151:5088-5095 (1993).

The ability of the PI3Kδ selective inhibitors to treat inflammatory glomerular injury can be demonstrated in a rat nephrotoxic serum nephritis model according to the method of Kawasaki et al., J. Immunol., 150:1074-1083 (1993).

The ability of the PI3Kδ selective inhibitors to treat radiation-induced enteritis can be demonstrated in a rat abdominal irradiation model according to the method of Panes et al., Gastroenterology, 108:1761-1769 (1995).

The ability of the PI3Kδ selective inhibitors to treat radiation pneumonitis can be demonstrated in a murine pulmonary irradiation model according to the method of Hallahan et al., Proc. Natl. Acad. Sci (USA), 94:6432-6437 (1997).

The ability of the PI3Kδ selective inhibitors to treat reperfusion injury can be demonstrated in the isolated heart according to the method of Tamiya et al., Immunopharmacology, 29:53-63 (1995), or in the anesthetized dog according to the model of Hartman et al., Cardiovasc. Res. 30:47-54 (1995).

The ability of the PI3Kδ selective inhibitors to treat pulmonary reperfusion injury can be demonstrated in a rat lung allograft reperfusion injury model according to the method of DeMeester et al., Transplantation, 62:1477-1485 (1996), or in a rabbit pulmonary edema model according to the method of Horgan et al., Am. J. Physiol. 261:H1578-H1584 (1991).

The ability of the PI3Kδ selective inhibitors to treat stroke can be demonstrated in a rabbit cerebral embolism stroke model according to the method of Bowes et al., Exp. Neurol., 119:215-219 (1993), in a rat middle cerebral artery ischemia-reperfusion model according to the method of Chopp et al., Stroke, 25:869-875 (1994), or in a rabbit reversible spinal cord ischemia model according to the method of Clark et al., Neurosurg., 75:623-627 (1991). The ability of the PI3Kδ selective inhibitors to treat cerebral vasospasm can be demonstrated in a rat experimental vasospasm model according to the method of Oshiro et al., Stroke, 28:2031-2038 (1997).

The ability of the PI3Kδ selective inhibitors to treat peripheral artery occlusion can be demonstrated in a rat skeletal muscle ischemia/reperfusion model according to the method of Gute et al., Mol. Cell Biochem., 179:169-187 (1998).

The ability of the PI3Kδ selective inhibitors to treat graft rejection can be demonstrated in a murine cardiac allograft rejection model according to the method of Isobe et al., Science, 255:1125-1127 (1992), in a murine thyroid gland kidney capsule model according to the method of Talento et al., Transplantation, 55:418422 (1993), in a cynomolgus monkey renal allograft model according to the method of Cosimi et al., J. Immunol., 144:4604-4612 (1990), in a rat nerve allograft model according to the method of Nakao et al., Muscle Nerve, 18:93-102 (1995), in a murine skin allograft model according to the method of Gorczynski and Wojcik, J. Immunol. 152:2011-2019 (1994), in a murine corneal allograft model according to the method of He et al., Opthalmol. Vis. Sci., 35:3218-3225 (1994), or in a xenogeneic pancreatic islet cell transplantation model according to the method of Zeng et al., Transplantation, 58:681-689 (1994).

The ability of the PI3Kδ selective inhibitors to treat graft-versus-host disease (GVHD) can be demonstrated in a murine lethal GVHD model according to the method of Harning et al., Transplantation, 52:842-845 (1991).

The ability of the PI3Kδ selective inhibitors to treat cancers can be demonstrated in a human lymphoma metastasis model (in mice) according to the method of Aoudjit et al., J. Immunol., 161:2333-2338 (1998).

As previously described, the term “PI3Kδ selective inhibitor” generally refers to a compound that inhibits the activity of the PI3Kδ isozyme more effectively than other isozymes of the PI3K family. The relative efficacies of compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a PI3Kδ selective inhibitor alternatively can be understood to refer to a compound that exhibits a 50% inhibitory concentration (IC₅₀) with respect to PI3Kδ that is at least 10-fold, in another aspect at least 20-fold, and in another aspect at least 30-fold, lower than the IC₅₀ value with respect to any or all of the other class I PI3K family members. In an alternative embodiment of the invention, the term PI3Kδ selective inhibitor can be understood to refer to a compound that exhibits an IC₅₀ with respect to PI3Kδ that is at least 50-fold, in another aspect at least 100-fold, in an additional aspect at least 200-fold, and in yet another aspect at least 500-fold, lower than the IC₅₀ with respect to any or all of the other PI3K class I family members. A PI3Kδ selective inhibitor is typically administered in an amount such that it selectively inhibits PI3Kδ activity, as described above.

Any selective inhibitor of PI3Kδ activity, including but not limited to small molecule inhibitors, peptide inhibitors, non-peptide inhibitors, naturally occurring inhibitors, and synthetic. inhibitors, may be used in the methods. Suitable PI3Kδ selective inhibitors have been described in U.S. Patent Publication 2002/161014 to Sadhu et al. and Knight et al., Bioorganic & Medicinal Chemistry, 12:4749-4759 (2004), the entire disclosures of which are hereby incorporated herein by reference. Compounds that compete with a PI3Kδ selective inhibitor compound described herein for binding to PI3K≢ and selectively inhibit PI3Kδ are also contemplated for use in the methods of the invention. Methods of identifying compounds which competitively bind with PI3Kδ, with respect to the PI3Kδ selective inhibitor compounds specifically provided herein, are well known in the art [see, e.g., Coligan et al., Current Protocols in Protein Science, A.5A.15-20, vol.3 (2002)]. In view of the above disclosures, therefore, PI3Kδ selective inhibitor embraces the specific PI3Kδ selective inhibitor compounds disclosed herein, compounds having similar inhibitory profiles, and compounds that compete with the such PI3Kδ selective inhibitor compounds for binding to PI3Kδ, and in each case, conjugates and derivatives thereof.

The methods of the invention may be applied to cell populations in vivo or ex vivo. “In vivo” means within a living individual, as within an animal or human. In this context, the methods of the invention may be used therapeutically or prophylactically in an individual, as described infra.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. For example, the invention may be used ex vivo to determine the optimal schedule and/or dosing of administration of a PI3Kδ selective inhibitor for a given indication, cell type, individual, and other parameters. Information gleaned from such use may be used for experimental or diagnostic purposes or in the clinic to set protocols for in vivo treatment. Other ex vivo uses for which the invention may be suited are described below or will become apparent to those skilled in the art.

The methods in accordance with the invention may include administering a PI3Kδ selective inhibitor with one or more other agents that either enhance the activity of the inhibitor or compliment its activity or use in treatment. Such additional factors and/or agents may produce an augmented or even synergistic effect when administered with a PI3Kδ selective inhibitor, or minimize side effects.

In one embodiment, the methods of the invention may include administering formulations comprising a PI3Kδ selective inhibitor of the invention with a particular cytokine, lymphokine, other hematopoietic factor, thrombolytic or anti-thrombotic factor, or anti-inflammatory agent before, during, or after administration of the PI3Kδ selective inhibitor. One of ordinary skill can easily determine if a particular cytokine, lymphokine, hematopoietic factor, thrombolytic or anti-thrombotic factor, and/or anti-inflammatory agent enhances or compliments the activity or use of the PI3Kδ selective inhibitors in treatment.

More specifically, and without limitation, the methods of the invention may comprise administering a PI3Kδ selective inhibitor with one or more of TNF, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Compositions in accordance with the invention may also include other known angiopoietins such as Ang-2, Ang-4, and Ang-Y, growth factors such as bone morphogenic protein-1, bone morphogenic protein-2, bone morphogenic protein-3, bone morphogenic protein-4, bone morphogenic protein-5, bone morphogenic protein-6, bone-morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β1, transforming growth factor β5, latent transforming growth factor β1, transforming growth factor β binding protein I, transforming growth factor β binding. protein II, transforming growth factorβ binding protein III, tumor necrosis factor receptor type I, tumor necrosis factor receptor type II, urokinase-type-plasminogen activator receptor, and chimeric proteins and biologically or immunologically active fragments thereof.

Methods of the invention contemplate use of PI3Kδ selective inhibitor compound having formula (I) or pharmaceutically acceptable salts and solvates thereof:

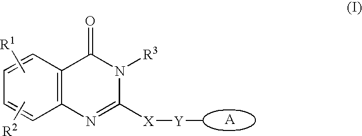

wherein A is an optionally substituted monocyclic or bicyclic ring system containing at least two nitrogen atoms, and at least one ring of the system is aromatic;

X is selected from the group consisting of C(R^{b})₂, CH₂CHR^{b}, and CH═C(R^{b});

Y is selected from the group consisting of null, S, SO, SO₂, NH, O, C(═O), OC(═O), C(═O)O, and NHC(═O)CH₂S;

R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C1-4alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃-8heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

or R¹ and R² are taken together to form a 3- or 4-membered alkylene or alkenylene chain component of a 5- or 6-membered ring, optionally containing at least one heteroatom;

R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})2, C₁₋₄alkyleneOR^{a}, C1-4alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

As used herein, the term “alkyl” is defined as straight chained and branched hydrocarbon groups containing the indicated number of carbon atoms, typically methyl, ethyl, and straight chain and branched propyl and butyl groups. The hydrocarbon group can contain up to 16 carbon atoms, for example, one to eight carbon atoms. The term “alkyl” includes “bridged alkyl,” i.e., a C₆-C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except for containing a carbon-carbon double bond. “Cycloalkenyl” is defined similarly to cycloalkyl, except a carbon-carbon double bond is present in the ring.

The term “alkylene” is defined as an alkyl group having a substituent. For example, the term “C₁₋₃alkylenearyl” refers to an alkyl group containing one to three carbon atoms, and substituted with an aryl group.

The term “heteroC₁₋₃alkyl” is defined as a C₁₋₃alkyl group further containing a heteroatom selected from O, S, and NR^{a}. For example, —CH₂OCH₃ or —CH₂CH₂SCH₃. The term “arylheteroC₁₋₃alkyl” refers to an aryl group having a heteroC₁₋₃alkyl substituent.

The term “halo” or “halogen” is defined herein to include fluorine, bromine, chlorine, and iodine.

The term “aryl,” alone or in combination, is defined herein as a monocyclic or polycyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, and amino. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like. The terms “arylC₁₋₃ alkyl” and “heteroarylC₁₋₃ alkyl” are defined as an aryl or heteroaryl group having a C₁₋₃ alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, and amino. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

Alternatively, the PI3Kδ selective inhibitor may be a compound having formula (II) or pharmaceutically acceptable salts and solvates thereof:

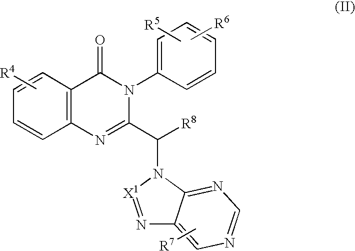

wherein R⁴, R⁵, R⁶, and R⁷, independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂Calkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈ cycloalkyl, C₁₋₈ heterocycloalkyl, arylOC₁₋₃alkyleneN(R^{a})₂, arylOC(═O)R^{b}, NHC(═O)C₁₋₃ alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R⁸ is selected from the group consisting of hydrogen, C₁₋₆alkyl, halo, CN, C(═O)R^{a}, and C(═O)OR^{a};

X¹ is selected from the group consisting of CH (i.e., a carbon atom having a hydrogen atom attached thereto) and nitrogen;

R^{a} is selected from the group consisting of hydrogen, C₁₋₆alkyl, C₃₋₈cycloalkyl, C₃₋₈heterocycloalkyl, C₁₋₃alkyleneN(R^{c})2,aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

The PI3Kδ selective inhibitor may also be a compound having formula (III) or pharmaceutically acceptable salts and solvates thereof:

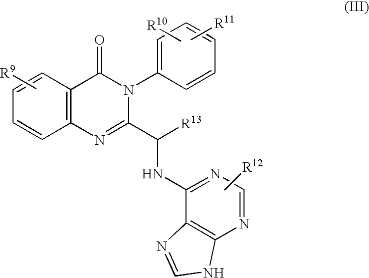

wherein R⁹, R¹⁰, R¹¹, and R¹², independently, are selected from the group consisting of hydrogen, amino, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenylenN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂), NR^{a}(SO₂C₁₋₄alkyl) (SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R¹³ is selected from the group consisting of hydrogen, C₁₋₆alkyl, halo, CN, C(═O)R^{a}, and C(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

More specifically, representative PI3Kδ selective inhibitors in accordance with the foregoing chemical formulae include but are not limited to 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chlorophenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9 H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenylydimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydro-quinazolin-2-ylmethyl ester; N-[3-(2-chlorophenyl)5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}-5-methyl-2-(9H-purin-6-ylsulfanylmethyl)-3H-quinazolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylrmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3,5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimid in-3-yl )-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; and 2-[1-(2-amino-9H-purin-6-ylamino)-ethyl]-5chloro-3-(3-fluoro-phenyl)-3H-quinazolin-4-one. Where a stereocenter is present, the methods can be practiced using a racemic mixture of the compounds or a specific enantiomer. In preferred embodiments where a stereocenter is present, the S-enantiomer of the above compounds is utilized. However, the methods of the invention include administration of all possible stereoisomers and geometric isomers of the aforementioned compounds.

Additionally, the methods include administration of PI3Kδ selective inhibitors comprising an arylmorpholine moiety [Knight et al., Bioorganic & Medicinal Chemistry, 12:47494759 (2004)]. Representative PI3Kδ selective inhibitors include but are not limited to 2-morpholin-4-yl-8-o-toxyloxy-1H-quinolin-4-one; 9-bromo-7-methyl-2-morpholin-4-yl-pyrido(1,2-a)-pyrimidin-4-one; 9-benzylamino-7-methyl-2-morpholin-4-yl-pyrido-(1,2 a)pyrimidin-4-one; 9-(3-amino-phenyl)-7-methyl-2-morpholin-4-yl-pyrido[1,2-a]pyrimidin-4-one; 9-(2-methoxy-phenylamino)-7-methyl-2-morpholin-4-yl-pyrido(1,2-a)pyrimidin-4-one; 7-methyl-2-morpholin-4-yl-9-o-tolylamino-pyrido(1,2-a)pyrimidin-4-one; 9-(3,4-dimethyl-phenylamino)-7-methyl-2-morpholin-4-yl-pyrido(1,2-a)pyrimidin-4-one; 7-methyl-9-(3-methyl-benzylamino)-2-morpholin-4-yl-pyrido(1,2-a)pyrimidin-4-one; 9-(2,3-dimethyl-phenylamino)-7-methyl-2-morpholin-4-yl-pyrido(1,2-a)pyrimidin-4-one; 7-methyl-9-(2-methyl-benzylamino)-2-morpholin-4-yl-pyrido(1,2-a) pyrimidin-4-one; 5-morpholin-4-yl-2-nitro-phenylamine; 1-(2-hydroxy-4-morpholin-4-yl-phenyl)-phenyl-methanone; and, 2-chloro-1-(2-hydroxy-4-morpholin-4-yl-phenyl)-ethanone.

Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma et al. (Eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Additionally, compounds that selectively negatively regulate p110δ mRNA expression more effectively than they do other isozymes of the PI3K family, and that possess acceptable pharmacological properties are contemplated for use as PI3Kδ selective inhibitors in the methods of the invention. Polynucleotides encoding human p110δ are disclosed, for example, in Genbank Accession Nos. AR255866, NM 005026, U86453, U57843 and Y10055, the entire disclosures of which are incorporated herein by reference [see also, Vanhaesebroeck et al., Proc. Natl. Acad. Sci., 94:4330-4335 (1997), the entire disclosure of which is incorporated herein by reference]. Representative polynucleotides, encoding mouse p110δ are disclosed, for example, in Genbank Accession Nos. BC035203, AK040867, U86587, and NM_{—}008840, and a polynucleotide encoding rat p110δ is disclosed in Genbank Accession No. XM_{—}345606, in each case the entire disclosures of which are incorporated herein by reference.

In one embodiment, the invention provides methods using antisense oligonucleotides which negatively regulate p110δ expression via hybridization to messenger RNA (mRNA) encoding p110δ. Suitable antisense oligonucleotide molecules are disclosed in U.S. Pat. No. 6,046,049, the entire disclosure of which is incorporated herein by reference. In one aspect, antisense oligonucleotides at least 5 to about 50 nucleotides in length, including all lengths (measured in number of nucleotides) in between, which specifically hybridize to mRNA encoding p110δ and inhibit mRNA expression, and as a result p110δ protein expression, are contemplated for use in the methods of the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides that are not perfectly complementary, i.e., those which include a limited number of mismatches with respect to a region in the target polynucleotide, also retain high degrees of hybridization specificity and therefore also can inhibit expression of the target mRNA. Accordingly, the invention contemplates methods using antisense oligonucleotides that are perfectly complementary to a target region in a polynucleotide encoding p110δ, as well as methods that utilize antisense oligonucleotides that are not perfectly complementary (i.e., include mismatches) to a target region in the target polynucleotide to the extent that the mismatches do not preclude specific hybridization to the target region in the target polynucleotide. Preparation and use of antisense compounds is described, for example, in U.S. Pat. No. 6,277,981, the entire disclosure of which is incorporated herein by reference [see also, Gibson (Ed.), Antisense and Ribozyme Methodology,,(1997), the entire disclosure of which is incorporated herein by reference].

The invention further contemplates methods utilizing ribozyme inhibitors which, as is known in the art, include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The methods of the invention therefore contemplate ribozyme inhibitors comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target p110δ-encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250, 6,410,224, 5,225,347; the entire disclosures of which are incorporated herein by reference.

The invention also contemplates use of methods in which RNAi technology is utilized for inhibiting p110δ expression. In one aspect, the invention provides double-stranded RNA (dsRNA) wherein one strand is complementary to a target region in a target p110δ-encoding polynucleotide. In general, dsRNA molecules of this type are less than 30 nucleotides in length and referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target p110δ-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. patent application Ser. No. 20040023390, the entire disclosure of which is incorporated herein by reference.

The invention further contemplates methods wherein inhibition of p110δ is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target p110δ-encoding polynucleotide. Because RNA lassos are circular and form tight topological linkage with the target region, inhibitors of this type are generally, not displaced by helicase action unlike typical antisense oligonucleotides, and therefore can be utilized as dosages lower than typical antisense oligonucleotides. Preparation and use of RNA lassos is described in U.S. Pat. No. 6,369,038, the entire disclosure of which is incorporated herein by reference.

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos: 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

Derivatization with bifunctional agents is useful for cross-linking a compound of the invention to a support matrix or to a carrier. One such carrier is polyethylene glycol (PEG). The PEG group may be of any convenient molecular weight and may be straight chain or branched. The average molecular weight of the PEG can range from about 2 kDa to about 100 kDa, in another aspect from about 5 kDa to about 50 kDa, and in a further aspect from about 5 kDa to about 10 kDa. The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, ci-haloacetyl, maleimido or hydrazino group) to a reactive group on the target inhibitor compound (e.g., an aldehyde, amino, ester, thiol, α-haloacetyl, maleimido or hydrazino group). Cross-linking agents can include, e.g., esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for inhibitor immobilization.

The pharmaceutical compositions of the invention may also include compounds derivatized to include one or more antibody Fc regions. Fc regions of antibodies comprise monomeric polypeptides that may be in dimeric or multimeric forms linked by disulfide bonds. or by non-covalent association. The number of intermolecular disulfide bonds between monomeric subunits of Fc molecules can be from one to four depending on the class (e.g., IgG, IgA, IgE) or subclass (e.g., IgG1, IgG2, IgG3, IgA1, IgGA2) of antibody from which the Fc region is derived. The term “Fc” as used herein is generic to the monomeric, dimeric, and multimeric forms of Fc molecules, with the Fc region being a wild type structure or a derivatized structure. The pharmaceutical compositions of the invention may also include the salvage receptor binding domain of an Fc molecule as described in WO 96/32478, as well as other Fc molecules described in WO 97/34631.

Such derivatized rhoieties preferably improve one or more characteristics of the inhibitor compounds of the invention, including for example, biological activity, solubility, absorption, biological half life, and the like. Alternatively, derivatized moieties result in compounds that have the same, or essentially the same, characteristics and/or properties of the compound that is not derivatized. The moieties may alternatively eliminate or attenuate any undesirable side effect of the compounds and the like.

Methods include administration of an inhibitor by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl and propylhydroxybenzoate, talc, alginates, carbohydrates, especially mannitol, α-lactose, anhydrous lactose, cellulose, sucrose, dextrose, sorbitol, modified dextrans, gum acacia, and starch. Some commercially available diluents are Fast-Flo, Emdex, STA-Rx 1500, Emcompress and Avicell. Such compositions may influence the physical state, stability, rate of in vivo release, and rate of in vivo clearance of the PI3Kδ inhibitor compounds [see, e.g., Remington's Pharmaceutical Sciences, 18th Ed. pp. 1435-1712 (1990), which is incorporated herein by reference].

Pharmaceutically acceptable fillers can include, for example, lactose, microcrystalline cellulose, dicalcium phosphate, tricalcium phosphate, calcium sulfate, dextrose, mannitol, and/or sucrose.

Inorganic salts including calcium triphosphate, magnesium carbonate, and sodium chloride may also be used as fillers in the pharmaceutical compositions. Amino acids may be used such as use in a buffer formulation of the pharmaceutical compositions.

Disintegrants may be included in solid dosage formulations of the inhibitors. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the formulation of the therapeutic ingredient to prevent sticking during the formulation process. Lubricants may be used as a layer between the therapeutic ingredient and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the therapeutic ingredient during formulation and to aid rearrangement during compression might be added. Suitable glidants include starch, talc, pyrogenic silica and hydrated silicoaluminate.

To aid dissolution of the therapeutic into the aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil, 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulation may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

In the methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

Also contemplated herein is pulmonary delivery of the PI3Kδ inhibitors in accordance with the invention. According to this aspect of the invention, the inhibitor is delivered to the lungs of a mammal while inhaling and traverses across the lung epithelial lining to the blood stream.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

When used in pulmonary administration methods, the inhibitors of the invention are most advantageously prepared in particulate form with an average particle size of less than 10 μm (or microns), for example, 0.5 to 5 μm, for most effective delivery to the distal lung.

Formulations suitable for use with a nebulizer, either jet or ultrasonic, will typically comprise the inventive compound dissolved in water at a concentration range of about 0.1 to 100 mg of inhibitor per mL of solution, 1 to 50 mg of inhibitor per mL of solution, or 5 to 25 mg of inhibitor per mL of solution. The formulation may also include a buffer. The nebulizer formulation may also contain a surfactant, to reduce or prevent surface induced aggregation of the inhibitor caused by atomization of the solution in forming the aerosol.

Formulations for use with a metered-dose inhaler device will generally comprise a finely divided powder containing the inventive inhibitors suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent or diluent such as lactose, sorbitol, sucrose, mannitol, trehalose, or xylitol in amounts which facilitate dispersal of the powder from the device, e.g., 50 to 90% by weight of the formulation.

Nasal delivery of the inventive compound is also contemplated. Nasal delivery allows the passage of the inhibitor to the blood stream directly after administering the therapeutic product to the nose, without the necessity for deposition of the product in the lung. Formulations for nasal delivery may include dextran or cyclodextran. Delivery via transport across other mucous membranes is also contemplated.

Toxicity and therapeutic efficacy of the PI3Kδ selective compounds can be determined by standard pharmaceutical procedures in cedl cultures or experimental animals, e.g., for determining the LD50 (the dose lethal to 50% of the population) and the ED50 (the dose therapeutically effective in 50% of the population). Additionally, this information can be determined in cell cultures or experimental animals additionally treated with other therapies including but not limited to radiation, chemotherapeutic agents, photodynamic therapies, radiofrequency ablation, anti-angiogenic agents, and combinations thereof.

In practice of the methods of the invention, the pharmaceutical compositions are generally provided in doses ranging from 1 pg compound/kg body weight to 1000 mg/kg, 0.1 mg/kg to 100 mg/kg, 0.1 mg/kg to 50 mg/kg, and 1 to 20 mg/kg, given in daily doses or in equivalent doses at longer or shorter intervals, e.g., every other day, twice weekly, weekly, or twice or three times daily. The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage [see, for example, Remington's Pharmaceutical Sciences, pp. 1435-1712, the disclosure of which is hereby incorporated by reference]. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the, above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various diseases and conditions capable of being treated with the methods of the invention.

### EXAMPLES

The following examples are provided to illustrate the invention, but are not intended to limit the scope thereof. Example 1 provides some of the reagents used in Examples 2-8. Examples 2-8 provide in vivo and in vitro evidence that PI3Kδ plays a prominent role in leukocyte accumulation in animal models of inflammation and that PI3Kδ selective inhibitors reduce leukocyte accumulation. More specifically, the examples provide evidence that PI3Kδ is present in endothelial, cells and contributes to leukocyte accumulation not only by participating in leukocyte transmigration to specific chemoattractants, but also in the ability of cytokine (e.g., TNFα stimulated endothelium to mediate effective adhesion/capturing of leuokocytes in flow.

### Example1

### Reagents

Monoclonal antibodies (mAb) and cell lines used in experiments included the ICAM-1 mAb RR 1/1 (biosource International, Camarillo, Calif.), FITC-conjugated goat F(ab′)₂ anti-mouse Ig (CALTAG Laboratories, Burlingame, Calif.), E-selectin mAb CL3 (ATCC, Manassas, Va.), FITC-conjugated Gr-1 (BD PharMingen, Franklin Lakes, N.J.), anti-Akt, PDK1, and PI3Kδ (Santa Cruz, Calif.), horseradish peroxidase-conjugated secondary antibodies (Jackson ImmunoResearch Laboratories Inc., West Grove, Pa.), CHO-ICAM-1 cells (ATCC, Manassas, Va.). Inflammatory agents and chemoattractants used included murine recombinant TNFα (PeproTech, Inc., Rocky Hill, N.J.), human recombinant TNFα (R&D Systems, Minneapolis, Minn.), LTB₄ (BIOMOL, Plymouth Meeting, Pa.), and fMLP (Sigma, St. Louis, Mo.). A small molecule selective PI3Kδ inhibitor in accordance with the invention, and recombinant PI3Kδ proteins were synthesized and purified as described by Sadhu et al., J. Immunol., 170:2647-2654 (2003).

### Example2

### The Role of PI3Kδ in Promoting Leukocyte-Endothelial Interactions In Vivo

To determine if PI3Kδ contributes to leukocyte accumulation in inflamed tissues such as lung tissue, the ability of leukocytes to interact with cytokine-stimulated endothelial cells in microvessels in the cremaster muscle of mice and to transmigrate was examined. Animals heterozygous for GFP expression under the murine lysozyme M locus control, which rendered neutrophils and other granulocytes visible by epifluorescence intravital microscopy, were used to quantitate leukocyte interactions with the vessel wall.

Mice in which green fluorescent protein (GFP) was knocked into the lysozyme M locus or the PI3Kδ catalytic subunit was deleted were generated as previously described [Faust et al., Blood, 96:719-726 (2000); and, Clayton et al., J. Exp. Med., 196:753-763 (2002)]. Subsequent matings were performed to yield mice that were heterozygous for GFP expression but deficient in PI3Kδ expression (mixed 129/Sv-C57BL/6 background) (GFP^{+/-}/PI3Kδ^{-/-} animals). All animals were handled in accordance with policies administered by institutional Animal Care and Use Committees.

The surgical preparation of animals for all in vivo studies was performed using standard techniques [see, e.g., Coxon, Immunity, 5:653-666 (1996)]. The cremaster muscle (CM) in GFP^{+/-} or GFP^{+/-}/PI3Kδ^{-/-} animals was inflamed with an intrascrotal injection of murine recombinant TNFα (20 ng/mouse). 2.5 hours after TNFα injection, the tissue was surgically exposed and positioned over a circular glass coverslip (25 mm) on a custom-built plexiglass stage for viewing. The stage was then placed on an intravital, microscope (IV-500; Mikron instruments, San Diego, Calif.) equipped with a silicon-intensified camera (VE1000SIT; Dage mti, Michigan City, Ind.) and the tissue kept moist by superfusion with thermo-controled (37° C.) bicarbonate-buffered saline. GFP-expressing cells (predominantly neutrophils, also including fewer monocytes) were. visualized through X20 or X40 water immersion objectives (Acroplan, Carl Zeiss Inc.) by epifluorescence from a Xenon arc stroboscope (Chadwick Helmuth, El Monte, Calif.) as they passed through the venous microcirculation of the cremaster muscle. Rolling fraction was defined as the percentage of cells that interact with a given venule in the, total number of cells that enter that venule during the same time period. The sticking fraction was defined as the number of rolling cells that became stationary for >30s post-superfusion of the CM with LTB₄ (0.1 μM). Venular shear rates were determined from optical Doppler velocimeter measurements of centerline erythrocyte velocity. The extent of leukocyte transmigration was evaluated at 30 and 60 min after application of LTB₄. Video images were recorded using a Hi8 VCR (Sony, Boston, Mass.) and analysis of performed using a PC-based image analysis system [Doggett et al., Biophys. J., 83:194-205 (2002)].

Oral administration of a compound in accordance with the invention one hour prior to intrascrotal injection of TNFα significantly impaired interactions between circulating granulocytes and venular endothelium as compared to vehicle treatment alone in GFP^{+/-} animals. A reduction in leukocyte tethering was also observed in animals lacking the PI3Kδ catalytic subunit (GFP^{+/-}/PI3Kδ^{-/-} animals) under similar conditions. This observation indicates that the reduction in leukocyte tethering in the animals treated with the inhibitor of the invention may be attributed to inhibition of PI3Kδ activity.

Moreover, the inhibitor-induced blockade or genetic deletion of the PI3Kδ isoform in mice resulted in a similar decrease (>50%) in the number of fluorescent cells that were observed to attach and roll during a defined period of time as compared to vehicle treated or WT matched littermates, respectively. The reduction in cell adhesion in these animals was not due to inhibitor-induced leukopenia as the number of circulating neutrophils was similar in both the control and experimental groups (2,857.3±803 and 2,730.7±1132.6 for control and inhibitor treated animals, respectively). The absolute number of circulating neutrophils in animals deficient in PI3Kδ was 2,997.7±776.1 (n=8). Wall shear rates calculated for each vessel were comparable in vehicle and inhibitor treated mice, thus alterations in the hemodynamic flow can be ruled out as a potential mechanism for the observed differences in cell adhesion.

In addition to reducing the percentage of interacting cells, the duration of leukocyte adhesion was also significantly depressed. For example, in inhibitor-treated GFP^{+/-} animals mice, the majority of neutrophils rolled for <2 s before releasing from the vessel wall. By contrast, in the vehicle-treated GFP^{+/-} animals, greater than 75 percent of cells were observed to interact at least about three times longer (>6 s) with the endothelial surface. Furthermore, in GFP^{+/-} animals that were administered an inhibitor in accordance with the invention, mean rolling velocities of neutrophils on TNF□-inflamed venules were approximately 8-fold higher than the corresponding control group (40.5±12.5 μm/s versus 4.9±7.6 μm/s, respectively). The mean rolling velocities of neutrophils in animals treated in accordance with the invention were comparable to that observed in PI3Kδ deficient GFP^{+/-}/PI3Kδ^{-/-} animals (35.7±13.2 μm/s, n=5).

In animals treated with a compound in accordance with the invention, LTB₄-induced migration of neutrophils across inflamed microvessels was diminished despite the continued accumulation of neutrophils on the luminal surface of the vessel wall. In contrast, extensive neutrophil transmigration was observed in vehicle-treated animals.

Taken together, these data indicate that the ability of leukocytes to initially form adhesive contact with the inflamed vessel wall (i.e., tethering) is negatively impacted by selective inhibition or deletion of this catalytic subunit. The results indicate that PI3Kδ activity is required for leukocyte tethering and transmigration.

### Example 3

### PI3Kδ is Expressed in Endothelium

Western blot experiments were conducted in accordance with the following protocol to determine p110δ expression in a variety of cells. PI3Kδ protein expression and function had not previously been demonstrated in vascular endothelium.

HUVEC cells were washed three times in ice-cold PBS and then lysed on ice in 50 mM Tris-HCl (pH 7.4), 1% Triton X-100, 150 mM NaCl, 1 mM EDTA and a cocktail of inhibitors to serine and cysteine proteases (Complete™, Mini, Roch Applied Science, Ind.). Lysates were harvested by scraping. The cell debris was removed by centrifugation at 12,000×g for 15 min at 4° C. Recombinant p110α, β, γ, and δ proteins (20 ng/lane) and cell lysate (100 μg/lane) were electrophoresed in precast 8% polyacrylamide gels (Invitrogen Life Technologies, Carlsbad, Calif.), transferred electrophoretically to a polyvinylidene difluoride membranes (Immobilon-P, Millipore, Billerica, Mass.), and immunoblotted with primary and horseradish peroxidase-conjugated secondary antibodies (Jackson ImmunoResearch Laboratories Inc., West Grove, Pa.) [Sadhu et al., J. Immunol., 170:2647-2654 (2003)]. Bound antibody was detected by chemiluminescence using ECL plus Western blot detection system according to the manufacturer's instructions (Amersham Biosciences, Piscataway, N.J.).

This Western blot analysis established that the p110δ catalytic subunit is expressed in endothelial cells.

### Example 4

### Intracellular Effects of p110δ Inhibition in Endothelial Cells

Treatment of HUVECs with a selective PI3Kδ inhibitor in accordance with the invention (2 μM) reduced TNFα-mediated signaling, as demonstrated by a reduction in phosphorylation of Akt, which is a downstream substrate for class I PI3Ks.

Quiescent HUVECs were pretreated with an inhibitor in accordance with the invention (2 or 10 μM) for 2 hours before stimulation with TNFα (0.1 to 50 ng/ml, usually 5 ng/ml) for a further 45 min [Madge et al., J. Biol. Chem., 275:15458-15465 (2000)]. Cell lysates were prepared as described above except that the lysis buffer also contained phosphatase inhibitors, 2 μM microcystin LR, 10 mM NaF, 1 mM Na₃VO₄, and 1 mM , β-glycerophosphate. Electroblots were analyzed for Akt activation (see discussion of Akt phosphorylation below) by Western blot analysis of total and phosphorylated Akt using specific antibodies.

Phosphorylation of Akt has been widely used as an indirect measure of PI3K activity in multiple cell types including HUVECs [Shiojima et al., Circ. Res., 90:1243-1250 (2002); Kandel et al., Exp. Cell Res., 253:210-229 (1999); and, Cantley et al., Science, 296:1655-1657 (2002)]. Broad inhibition of class Ia PI3Ks in endothelium with LY294002 has been shown to reduce phosphorylation of Akt in response to TNF [Madge et al., J. Biol. Chem., 275:15458-15465 (2000)].

Further evidence that suggests that compounds of the invention inhibit PI3Kδ function in endothelial cells rather than a down stream effector molecule involved in Akt phophorylation, is provided by direct measurement of the activity of PDK1 immunoprecipitated from TNFα-stimulated HUVECs pretreated with compound or vehicle control. Incubation of intact HUVECs, but not their lysates, with compound reduced the kinase activity of this pleckstrin homology domain containing protein in response to TNFα. Thus, PI3Kδ activity is required for PDK1 and Akt function in endothelium as previously described for neutrophils.

The selective inhibitors of the invention do not significantly block additional intracellular signaling pathways (e.g., p38 MAPK or insulin receptor tyrosine kinase) that are also critical for general cell function and survival. (See Table 1; see also Sadhu et al., J. Immunol., 170:2647-2654 (2003)).

TABLE 1
The effect of an inhibitor in accordance with the
invention (10 μM) on the activity of several protein
kinases and a phosphatase.

| Enzyme | Activity (% of control) ± SD |
|---|---|
| EGE receptor tyrosine kinase | 102 ± 5.5 |
| Insulin receptor tyrosine kinase | 98 ± 6.2 |
| CD45 tyrosine phosphatase | 104 ± 2.2 |
| PKC-θ | 97 ± 5.5 |
| PDK1 | 91.5 ± 2.1 |
| Lck | 116.5 ± 9.2 |
| P70S6K | 98.5 ± 0.7 |
| CDK2/cyclinA | 92.5 ± 2.12 |
| ZAP-70 | 97.5 ± 13.4 |
| p38 MAPK | No inhibition* |
| DNA-PK | No inhibition* |
| CHK1 | No inhibition* |
| cSrc | No inhibition* |
| CK1 | No inhibition* |
| PKBα (Akt 1) | No inhibition* |
| PKCα | No inhibition* |
| PKCβII | No inhibition* |

Protein kinase assays were performed in the presence of 100 μM ATP. The kinase activities marked with an asterisk were reported by Sadhu et al., J. Immunol., 170:2647-2654 (2003).

### Example 5

### Inhibition of PI3Kδ activity in endothelial cells inhibits initial adhesion of leukocytes to endothelial cells

Inhibition of PI3Kδ activity in either endothelium or neutrophils could potentially account for the observed reduction in adhesive interactions between these two cell types in vivo. See Examples 2 and 8. To determine whether PI3Kδ activity in endothelium or leukocytes was the key component in regulating leukocyte adhesion in flow, human and murine neutrophil binding to a HUVEC or bEND3.1 monolayer, respectively, were evaluated using a parallel plate flow chamber apparatus.

First, the effect of inhibiting PI3Kδ in endothelial cells was examined. Human umbilical vein endothelial cells (HUVECS) (3-4 passages; Cambrex Inc., East Rutherford, N.J.) grown on fibronectin-coated glass cover slips were pretreated with an inhibitor in accordance-with the invention (2 μM) or vehicle control for 1 hour prior-to being stimulated with TNFα (5 ng/ml, 4 h). Stimulation with TNFα induces expression of E-selectin by the endothelial cells. Peripheral blood neutrophils from healthy volunteers were isolated from whole blood by dextran sedimentation followed by density separation over Ficoll-Hypaque and hypotonic lysis. Approval was obtained from the Washington University Institutional Review Board for these studies. Informed consent was provided according to the Declaration of Helsinki. Neutrophils (1×10⁶/ml; HBSS, 10 mM HEPES, 1 mM CaCl₂, 0.5% HSA, pH 7.4) were infused over the endothelial cell monolayer that was incorporated into a parallel plate flow chamber (GlycoTech, Rockville, Md.). for 5 min at shear rates of 100 and 300 s⁻¹. The percentage of neutrophils that attached to TNFα-stimulated HUVECs treated with an inhibitor in accordance with the invention versus control treated (vehicle alone, 0.3% DMSO) TNFα-stimulated HUVECs was determined.

In comparison to neutrophil tethering to HUVECs treated with vehicle alone, neutrophil tethering to HUVECs pre-incubated with an inhibitor according to the invention was reduced by 28% and 40% at physiological wall shear rates of 100 and 300 s⁻¹, respectively. Thus, inhibition of PI3Kδ activity in endothelial cells does reduce in adhesive interactions between the two cell types.

Next, the effect of inhibiting PI3Kδ in neutrophils was examined. Purified neutrophilic polymorphonuclear granulocytes (PMNs) (1×10⁶/ml; HBSS, 10 mM HEPES, 1 mM CaCl2, 0.5% HSA, pH 7.4) from mouse bone marrow (BM) were infused over a monolayer of TNFα-activated mouse endothelioma cells derived from brain capillaries (bEND3.1 cells) grown to confluence on fibronectin-coated glass coverslips. Mouse BM PMNs were isolated from femurs and tibias obtained from PI3Kδ deficient mice and, wild-type (WT) littermate controls by density centrifugation as previously described (Roberts et al., Immunity, 10:183-196 (1999); Lowell et al., J. Cell Biol., 133:895-910 (1996)). Briefly, cells were flushed from the marrow using Ca²⁺ and Mg²⁺-free Hank's balanced salt solution (HBSS, Sigma) supplemented with 0.2% buffer saline (BSA), and washed, after which neutrdphils were isolated using a discontinuous Percoll (Pharmacia, Piscataway, N.J.) gradient. Red cell depletion was performed using density centrifugation in Ficoll (density 1.119; 30 min at 1200×g). The resulting cell populations in both genotypes were equivalent for expression of the granulocyte marker Gr-1 (79% to 84% positive). The number of interacting PMNs was determined after 5 min of flow (1 dyn/cm²) and expressed per unit area of the field of view.

In contrast to treatment of endothelial cells with an inhibitor according to the invention, treatment of neutrophils with the identical concentration of inhibitor prior to their infusion over a HUVEC substrate pre-treated with only TNFα did not reduce neutrophil tethering. Moreover, no significant difference in attachment was noted for WT versus PI3Kδ deficient neutrophils interacting with the murine endothelioma cell line under identical flow conditions. These results are consistent with a previous study demonstrating that blockade of PI3K activity in neutrophils with wortmannin or LY294002 does not alter selectin-dependent adhesion [Constantin et al., Immunity, 13:759-769 (2000)].

In additional experiments where leukocytes were pre-treated with an inhibitor of the invention as described above, the HUVECS were pre-incubated with mAb CL3 (50 μg/ml, 15 min) to block E-selectin binding. Results showed that E-selectin contributed >80% of neutrophil tethering to TNFα-stimulated HUVECs. Endothelial cells therefore recruit leukocytes at least in part through selectins.

Thus, p110δ was found to-be present in endothelial cells and to participate in leukocyte tethering by modulating the proadhesive state of the endothelial cells in response to an inflammatory mediator such as TNFα.

### Example 6

### The Lack of Impact of PI3Kδ Inhibition on Firm Adhesion

In order for leukocyte transmigration to occur, engagement of the leukocyte integrins with ICAMs expressed on venular endothelium (“firm adhesion”) is necessary for leukocytes to stably adhere to the vessel wall (in addition to the requirement for selectin-mediated tethering and rolling) [Dunne et al., Blood, 99:336-341 (2002)]. To determine the role of PI3Kδ in firm adhesion, the ability of leukocytes-rolling on inflamed venular endothelium to undergo integrin-mediated firm adhesion in response to an activating stimulus was investigated in vivo.

When the inflamed cremaster muscle was superfused with LTB₄ in vivo, leukocytes rapidly transitioned from rolling to firm adhesion despite the presence of a PI3Kδ inhibitor in accordance with the invention. The inhibitor concentration was 12.8±3.7 μM (a mean plasma known to predominantly inhibit PI3Kδ activity) when LTB₄ was applied. Because firm adhesion requires the β₂-integrins (i.e., Mac-1 and LFA-1) and endothelial cell ICAM-1, these receptor-ligand pairs appear to not be significantly perturbed under these experimental conditions. These experiments were performed in accordance with the procedures described in Example 2.

To confirm that the ability of the integrins on the surface of leukocytes to bind to ICAMs was not significantly altered in the presence of an inhibitor in accordance with the invention, LTB₄-triggered firm adhesion to ICAM-1 was also evaluated in vitro. Purified neutrophils (2×10⁶/ml in HBSS buffer containing 2 mM MgCl₂) were incubated with 2 μM of a compound in accordance with the invention prior to conducting the adhesion assays. This concentration (2 μM) primarily inhibits PI3Kδ but not other class Ia or Ib PI3Ks. Treated neutrophils were then stimulated with LTB₄ (0.1 μM) and allowed to bind in stasis to CHO cells transfected with human ICAM-1 before subjecting them to physiological wall shear stresses of 2 and 4 dyn/cm². ICAM-1 expression on these cells was confirmed by-flow cytometry using mAb R 1/1 (fluorescence intensity >10³, data not shown). As in the in vivo experiments described above, PI3Kδ inhibition did not impair integrin-mediated firm adhesion. For example, more than 80% of LTB₄-stimulated neutrophils remained bound to the ICAM-1 substrate in the presence or absence of an inhibitor in accordance with the invention. The percentage of cells that remained adherent after 20 seconds (s),at each wall, shear stress was determined by off-line video analysis.

Thus, PI3Kδ appears to be involved in the regulation of E-selectin tethering (Example 5) but not β₂-integrin-mediated firm adhesion of neutrophils to vascular endothelium.

### Example 7

### The Role of PI3Kδ in Leukocyte Transmigration

The final step required for accumulation of leukocytes in inflamed tissues, transmigration, relies upon chemoattractant-directed migration, an event that is known to involve PI3Ks. A recent study suggested that PI3Kδ was involved in this process as treatment of neutrophils with a compound in accordance with the invention diminished fMLP-induced chemotaxis on an ICAM-1 substrate in vitro, in the absence of hemodynamic forces [Sadhu et al., J. Immunol., 170:2647-2654 (2003)].

Neutrophil chemotaxis experiments were conducted as described [Roth et al., J. Immunol. Methods, 188:97-116 (1995)]. Briefly, purified human neutrophils were incubated with DMSO (0.3% v/v) or an inhibitor in accordance with the invention reconstituted in DMSO (0.3%) for 20 minutes at room temperature. Cells were added to bare filter inserts (Transwell™ 5 μm pore size; Coming Costar, Cambridge, Mass.), that were placed into wells containing chemoattractants or control medium of a Ultra low 24-well cluster plate, and incubated for 1 hour at 37° C. in a 5% CO₂ humidified environment. The number of neutrophils that migrated into the bottom well was determined by FACScan (Becton Dickinson, San Jose, Calif.). Results were expressed as percent neutrophil migration relative to the control (medium without inhibitor).

A dose response curve was generated to determine the concentration of LTB₄ necessary to support half-maximal migration across a bare filter insert. Maximal transmigration for neutrophils purified from mouse bone marrow occurred between 100 to 250 nM of LTB₄. These data are consistent with previously published results. Tager et al., J Exp. Med., 192:439-46 (2000). Treatment of WT neutrophils with 2 μM inhibitor in accordance with the invention diminished migration in response to LTB₄ (30 nM) by ˜30%, a equivalent to that observed for PI3Kδ deficient cells. Preincubation of cells lacking this PI3K isoform, however, with the identical concentration of inhibitor had no further effects on chemotaxis suggesting its specificity towards p110δ.

These results demonstrate that the PI3Kδ isoform is involved in chemotaxis, but its impact is not restricted to reducing directed movement to the bacterial product, fMLP. For example, LTB₄-induced migration of neutrophils across inflamed microvessels was diminished in vivo in animals treated in accordance with the invention. See Example 2. LTB₄-induced neutrophil transmigration was reduced despite the continued accumulation of neutrophils on the luminal surface of the vessel wall. In contrast, extensive neutrophil transmigration was observed in vehicle-treated animals.

### Example 8

### PI3Kδ Activity Contributes to Leukocyte Accumulation in a Model of Acute Pulmonary Inflammation

An acute lung injury model was used to determine if the effects of PI3Kδ blockade on leukocyte accumulation in inflamed tissues are limited to a specific vascular bed or for that matter a particular species. This example demonstrates that PI3Kδ activity is required for chemoattractant-triggered leukocyte accumulation, specifically neutrophil accumulation, into the airway space.

Lewis rats to be treated with an inhibitor in accordance with the invention or vehicle control (PEG400) were first challenged with LPS [Asti et al., Pulm. Pharmacol. Ther., 13:61-69 (2000)]. Briefly, the trachea was exposed by standard surgical procedures and 100 μl saline solution or saline containing LPS (Escherichia Coli Serotype 0111:B4, Sigma) was instilled. Six hours following the challenge, rats were euthanized and the bronchoalveolar lavage (BAL) fluid was collected for cell differentials. Total white blood cell (WBC) and neutrophil counts were determined (Hemavet™ 850 FS cell counter). Cell populations were identified by morphological examination of smears prepared by cytocentrifugation.

Animals received a single oral dose of either a compound in accordance with the invention (25 mg/kg for mice and 20 or 40 mg/kg for rats) or vehicle (PEG400). Blood samples were subsequently drawn at indicated time points and plasma concentration of the compound determined after liquid-liquid extraction by LC/MS. The lower quantification limit was 50 ng/ml. Plasma samples from control animals (vehicle alone) were used as the blank control.

Whole blood (200 μl per well) was incubated with an inhibitor in accordance with the invention for 30 minutes at 37° C. and cells were stimulated with LPS (100 ng/ml) for 8 hours (h). The samples were centrifuged and the supernatant was collected and analyzed for TNFα by ELISA (Cayman Chemical Co., Ann Arbor Mich.). Results are expressed as the percentage TNFα released relative to control.

Instillation of LPS into the trachea of rats resulted in about a 100-fold increase in neutrophil counts in bronchoalveolar lavage (BAL) fluid six hours post-challenge as compared to PBS control.

Animals orally treated one hour prior to LPS challenge with either 20 mg or 40 mg of an inhibitor in accordance with the invention per kg of body weight had an approximately 60 to 80% reduction in the accumulation of neutrophils in BAL fluid, respectively. Importantly, inhibitor plasma levels were within the range that effectively blocked PI3Kδ biochemical activity but not the other class I isoforms of PI3K that are expressed in neutrophils [Sadhu et al., J. Immunol., 170:2647-2654 (2003)]. Despite this reduction in neutrophil influx, TNFα a cytokine essential for endothelial cell activation, was still detectable in BAL fluid of LPS-treated mice that received inhibitor in accordance with the invention. In addition, the inhibitors do not appear to be toxic to cells as neutrophils treated with inhibitors in accordance with the invention at concentrations as high as 50 μM remained >95% viable.

Numerous modifications and variations in the invention as set forth in the above illustrative examples are expected to occur to those skilled in the art. Consequently only such limitations as appear in the appended claims should be placed on the invention.

1. A method of inhibiting leukocyte accumulation, comprising:
selectively inhibiting phosphoinositide 3-kinase delta (PI3Kδ) activity in endothelial cells, thereby inhibiting leukocyte accumulation.

2. The method according to claim 1, wherein inhibiting comprises administering an amount of a phosphoinositide 3-kinase delta (PI3Kδ) selective inhibitor effective to inhibit p110 delta (p110δ) in endothelial cells.

3. The method according to claim 1, wherein said inhibiting is in vitro.

4. The method according to claim 1, wherein said inhibiting is performed in an individual in need thereof.

5. The method according to claim 1, wherein the leukocytes are selected from the group consisting of neutrophils, eosinophils, basophils, T-lymphocytes, B-lymphocytes, monocytes, macrophages, dendritic cells, Langerhans cells, and mast cells.

6. The method according to claim 1, wherein the leukocytes are neutrophils.

7. The method according to claim 1, wherein the leukocyte accumulation is mediated by selectin receptors.

8. The method according to claim 1, wherein the leukocyte accumulation is mediated by P-selectin receptors.

9. The method according to claim 1, wherein the leukocyte accumulation is mediated by E-selectin receptors.

10. The method according to claim 1, wherein a mean rolling velocity of the leukocytes on the endothelial cells is increased relative to a mean rolling velocity of leukocytes on endothelial cells wherein PI3Kδ activity has not been selectively inhibited.

11. The method according to claim 10, wherein the mean rolling velocity is increased by at least about 200 percent.

12. The method according to claim 1, wherein integrin-mediated leukocyte firm adhesion is not substantially inhibited.

13. The method according to claim 1, wherein AKT-activation of the endothelial cells is reduced relative to endothelial cells wherein PI3Kδ activity has not been selectively inhibited.

14. The method according to claim 1, wherein PDK1 enzyme activity of the endothelial cells is reduced relative to endothelial cells wherein PI3Kδ activity has not been selectively inhibited.

15. The method according to claim 1, wherein p110δ expression by the endothelial cells is reduced relative to endothelial cells wherein PI3Kδ activity has not been selectively inhibited.

16. The method according to claim 1, wherein the leukocyte accumulation is initiated in response to an inflammation mediator.

17. The method according to claim 16, wherein the inflammation mediator is selected from the group consisting of histamine, tumor necrosis factor alpha (TNF-alpha), interleukin 1 alpha (IL-1 alpha), interleukin 1 beta (IL-1 beta), Duffy antigen/receptor for chemokines (DARC), lymphotactin, stromal cell-derived factor-1 (SDF-1), transforming growth factor beta (TGF-beta), gamma-interferon (IFN-gamma), leukotriene B4 (LTB4), thrombin, formyl-methionyl-leucyl-phenylalanine (fMLP), lipopolysaccharides (LPS), platelet-activating factor (PAF), and lysophospholipids.

18. The method according to claim 16, wherein the inflammation mediator is selected from the group consisting of histamine, tumor necrosis factor alpha (TNF-alpha), and interleukin 1 alpha (IL-1 alpha), interleukin 1 beta (IL-1 beta), thrombin, and lipopolysaccharides (LPS).

19. The method according to claim 4, wherein the individual has an inflammatory condition selected from the group consisting of chronic inflammatory diseases, tissue or organ transplant rejections, graft versus host disease,(GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), asthma, lupus nephritis, Crohn's disease, ulcerative colitis, necrotising enterocolitis, pancreatitis, neumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

20. The method according to claim 2, wherein the PI3Kδ selective inhibitor is a compound having formula (I) or pharmaceutically acceptable salts and solvates thereof: 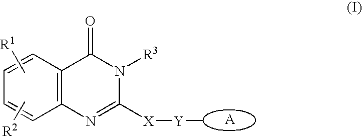
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆ alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂), OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO2N(R^{a})₂, N(R^{a})₂, C(═O)OR^{a}, NR^{a}SO₂CF₃, CN, NO₂, C(═O)Rₐ, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O )C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl,aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)ORₐ.

21. The method according to claim 2, wherein the PI3Kδ selective inhibitor is selected from the group consisting of:
2-(6-aminopurin-9-ylmethyl)-3-(2-chlorophenyl)-6,7-dimethoxy-3H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-6-chloro-3-(2-chlorophenyl)-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one,
5-chloro-3-(2-fluorophenyl)-2-(9H-purin-6-yl-sulfanylmethyl)-3H-quinazolin-4-one;
2-(6-aminopurin-9-ylmethyl )-5-chloro-3-(2-fluorophenyl )-3H-quinazolin-4-one;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophenyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl)-2-(9H-purin-6-yl-sulfanylmethyl)-3H-quinazolin-4-one;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl)-6,7-difluoro-2-(9H-purin-6-yl-sulfanylmethyl)-3H-quinazolin-4-one;
3-(2 chlorophenyl)-6-fluoro-2-(9H-purin-6-yl-sulfanylmethyl) 3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one;
2-(6-aminopurin-9-ylmethyl)-5-chloro-3-o-tolyl-3H-quinazolin-4-one;
2-(6-aminopurin-9-ylmethyl)-5-chloro-3-(2-methoxy-phenyl)-3H-quinazolin-4-one;
2-(2-amino-9H-purin-6-ylsulfanylmethyl)-3-cyclopropyl-5-methyl-3H-quinazolin-4-one;
3-cyclopropylmethyl-5-methyl-2-(9H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3H-quinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2-(9H-purin-6-ylsulfanylmethyl)-3H-quinazolin-4-one;
2-(6-aminopurin-9-ylmethyl)-3-cyclohexyl-5-methyl-3H-quinazolin-4-one;
2-(2-amino-9H-purin-6-ylsulfanylmethyl)-3-cyclo-hexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)-dimethylamino-(9H-purin-6-ylsulfanylmethyl)-3H-quinazolin-4-one;
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydro-quinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylaminopurin-9-ylmethyl)-5-methyl-3-o-tolyl-3H-quinazolin-4-one;
5-methyl-2-(2-methyl-6-oxo-1,6-dihydro-purin-7-ylmethyl)-3-o-tolyl-3H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl)-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1,3,5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-diamino-pyrimidin-4-ylsulfanylmethyl)-5-methyl-3-o-tolyl-3H-quinazolin-4-one;
5-methyl-2-(5-methyl-[1,2,4]triazolo[1,5-a]pyrimidin-7-ylsulfanylmethyl )-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo[4,5-d]pyrimidin-3-yl-methyl)-5-methyl-3-o-tolyl-3H-quinazolin-4-one;
2-(7-amino-1,2,3-triazolo[4 ,5-d] pyrimidin-1-yl-methyl )-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-3H-quinazolin-4-one;
2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one;
N-{2-[5-Methyl-4-oxo-2-(9H-purin-6-ylsulfanylmethyl)-4H-quinazolin-3-yl]-phenyl}-acetamide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid;
3-{2-[(2-dimethylaminoethyl)methylamino]phenyl}-5-methyl-2-(9H-purin-6-ylsulfanylmethyl)-3H-quin-azolin-4-one;
3-(2-chlorophenyl)-5-methoxy-2-(9H-purin-6-ylsulfanylmethyl)-3H-quinazolin-4-one;
3-(2-chlorophenyl-5-(2-morpholin-4-yl-ethylamino)-2-(9H-purin-6-ylsulfanylmethyl)-3H-quinazolin-4-one;
3-benzyl-5-methoxy-2-(9H-purin-6-ylsulfanylmethyl)-3H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[1-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2-(2-(1-methylpyrrolidin-2-yl)-ethoxy)-phenyl}-3H-quinazolin-4-one;
2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one;
2-{2-(1-(6-aminopurin-9-ylmethyl)-5-methyl-4-oxo-4H-quinazolin-3-yl]-phenoxy}-acetamide;
2-[(6-aminopurin-9-yl)methyl]-5-methyl-3-o-tolyl-3-hydroquinazolin-4-one;
3-(3,5-difluorophenyl)-5-methyl-2-[(purin-6-ylamino)methyl]-3-hydroquinazolin-4-one;
3-(2,6-dichlorophenyl)-5-methyl-2-[(purin-6-ylamino)methyl]-3-hydroquinazolin-4-one;
3-(2-Fluoro-phenyl)-2-[1-(2-fluoro-9H-purin-6-ylamino)-ethyl]-5-methyl-3-hydroquinazolin-4-one;
2-[1-(6-aminopurin-9-yl)ethyl]-3-(3,5-difluorophenyl)-5-methyl-3-hydroquinazolin-4-one;
2-[1-(7-Amino-[1,2,3]triazolo[4,5-d]pyrimidin-3-yl)-ethyl]-3-(3,5-difluoro-phenyl)-5-methyl-3H-quinazolin-4-one;
5-chloro-3-(3,5-difluoro-phenyl)-2-[1-(9H-purin-6-ylamino)-propyl]-3H-quinazolin-4-one;
3-phenyl-2-[1-(9H-purin-6-ylamino)-propyl]-3H-quinazolin-4-one;
5-fluoro-3-phenyl-2-[1-(9H-purin-6-ylamino)-propyl]-3H-quinazolin-4-one;
3-(2,6-difluoro-phenyl)-5-methyl-2-[1-(9H-purin-6-ylamino)-propyl]-3H-quinazolin-4-one;
6-fluoro-3-phenyl-2-[1-(9H-purin-6-ylamino)-ethyl]-3H-quinazolin-4-one;
3-(3,5-difluoro-phenyl)-5-methyl-2-[1-(9H-purin-6-ylamino)-ethyl]-3H-quinazolin-4-one;
5-fluoro-3-phenyl-2-[1-(9H-purin-6-ylamino)-ethyl]-3H-quinazolin-4-one;
3-(2,3-difluoro-phenyl)-5-methyl-2-[1-(9H-purin-6-ylamino)-ethyl]-3H-quinazolin-4-one;
5-methyl-3-phenyl-2-[1-(9H-purin-6-ylamino)-ethyl]-3H-quinazolin-4-one;
3-(3-chloro-phenyl)-5-methyl-2-[1-(9H-purin-6-ylamino)-ethyl]-3H-quinazolin-4-one;
5-methyl-3-phenyl-2-[(9H-purin-6-ylamino)-methyl]-3H-quinazolin-4-one;
2-[(2-amino-9H-purin-6-ylamino)-methyl]-3-(3,5-difluoro-phenyl)-5-methyl-3H-quinazolin-4-one;
3-{2-[(2-diethylamino-ethyl)-methyl-amino]-phenyl}-5-methyl-2-[(9H-purin-6-ylamino)-methyl]-3H-quinazolin-4-one;
5-chloro-3-(2-fluoro-phenyl)-2-[(9H-purin-6-ylamino)-methyl]-3H-quinazolin-4-one;
5-chloro-2-[(9H-purin-6-ylamino)-methyl]-3-o-tolyl-3H-quinazolin-4-one;
5-chloro-3-(2-chloro-phenyl)-2-[(9H-purin-6-ylamino)-methyl]-3H-quinazolin-4-one;
6-fluoro-3-(3-fluoro-phenyl)-2-[1-(9H-purin-6-ylamino)-ethyl]-3H-quinazolin-4-one;
2-[1-(2-amino-9H-purin-6-ylamino)-ethyl]-5-chloro-3-(3-fluoro-phenyl)-3H-quinazolin-4-one; and,
pharmaceutically acceptable salts and solvates thereof.

22. A method of inhibiting leukocyte tethering to endothelial cells, comprising:
selectively inhibiting phosphoinositide 3-kinase delta (PI3Kδ) activity in endothelial cells, thereby inhibiting leukocyte tethering to endothelial cells.

23. The method according to claim 22, wherein inhibiting comprises administering an amount of a phosphoinositide 3-kinase delta (PI3Kδ) selective inhibitor effective to inhibit leukocyte tethering to endothelial cells.

24. The method according to claim 22, wherein said inhibiting is in vitro.

25. The method according to claim 22, wherein said inhibiting is performed in an individual in need-thereof.

26. The method according to claim 22, wherein the leukocytes are selected from the group consisting of neutrophils, eosinophils, basophils, T-lymphocytes, B-lymphocytes, monocytes, macrophages, dendritic cells, Langerhans cells, and mast cells.

27. The method according to claim 22, wherein the leukocytes are neutrophils.

28. The method according to claim 22, wherein the leukocyte tethering to endothelial cells is mediated by selectin receptors.

29. The method according to claim 22, wherein the leukocyte tethering to endothelial cells is mediated by P-selectin receptors.

30. The method according to claim 22, wherein the leukocyte tethering to endothelial cells is mediated by E-selectin receptors.

31. The method according to claim 22, wherein a mean rolling velocity of the leukocytes on the endothelial cells is increased relative to a mean rolling velocity of leukocytes on endothelial cells wherein PI3Kδ activity has not been selectively inhibited.

32. The method according to claim 25, wherein the mean rolling velocity is increased by at least about 200 percent.

33. The method according to claim 22, wherein integrin-mediated leukocyte adhesion to endothelial cells is not substantially inhibited.

34. The method according to claim 22, wherein AKT-activation is reduced relative to endothelial cells wherein PI3Kδ activity has not been selectively inhibited.

35. The method according to claim 22, wherein PDK1 enzyme activity is reduced relative to endothelial cells wherein PI3Kδ activity has not been selectively inhibited.

36. The method according to claim 22, wherein p110δ expression by the endothelial cells is reduced relative to endothelial cells wherein PI3Kδ activity has not been selectively inhibited.

37. The method according to claim 22, wherein the leukocyte tethering to endothelial cells is initiated in response to an inflammation mediator.

38. The method according to claim 37, wherein the inflammation mediator is selected from the group consisting of histamine, tumor necrosis factor alpha (TNF-alpha), interleukin 1 alpha (IL-1 alpha), interleukin 1 beta (IL-1 beta), Duffy antigen/receptor for chemokines (DARC), lymphotactin, stromal cell-derived factor-1 (SDF-1), transforming growth factor beta (TGF-beta), gamma-interferon (IFN-gamma), leukotriene B4 (LTB4), thrombin, formyl-methionyl-leucyl-phenylalanine (fMLP), lipopolysaccharides (LPS), platelet-activating factor (PAF), and lysophospholipids.

39. The method according to claim 37, wherein the inflammation mediator is selected from the group consisting of histamine, tumor necrosis factor alpha (TNF-alpha), and interleukin 1 alpha (IL-1 alpha), interleukin 1 beta (IL-1 beta), thrombin, and lipopolysaccharides (LPS).

40. The method according to claim 25, wherein the individual has a inflammatory condition selected from the group consisting of chronic inflammatory diseases, tissue or organ transplant rejections, graft versus host, disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), asthma, lupus nephritis, Crohn's disease, ulcerative colitis, necrotising enterocolitis, pancreatitis, neumocystis carinii pneumonia (PCP), inflammatory bowel disease (IBD), severe-acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

41. The method according to claim 23, wherein the PI3Kδ selective inhibitor is a compound having formula (I) or pharmaceutically acceptable salts and solvates thereof: 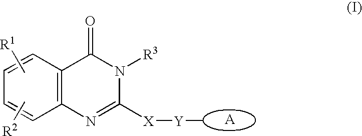
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R , NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO2N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C ₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully unsaturated, containing at least one, heteroatom selected from the group consisting of oxygen, nitrogen, and sulfur, and optionally substituted with C₁₋₄alkyl or C(═O)OR^{a}.

42. The method according to claim 23, wherein the PI3Kδ selective inhibitor is selected from the group consisting of:
2-(6-aminopurin-o-ylmethyl)-3-(2-chlorophenyl)-6,7-dimethoxy-3H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-6-chloro-3-(2-chlorophenyl)-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one;
5-chloro-3-(2-fluorophenyl)-2-(9H-purin-6-yl-sulfanylmethyl)-3H-quinazolin-4-one;
2-(6-aminopurin-9-ylmethyl)-5-chloro-3-(2-fluorophenyl)-3 H-quinazolin-4-one ;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophenyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl)-2-(9H-purin-6-yl-sulfanylmethyl)-3H-quinazolin-4-one;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl )-6,7-difluoro-2(9H-purin-6-yl-sulfanylmethyl )-3H-quinazolin-4-one;
3-(2-chlorophenyl)-6-fluoro-2-(9H-purin-6-yl-sulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one;
2-(6-aminopurin-9-ylmethyl)-5-chloro-3-o-tolyl-3H-quinazolin-4-one;
2-(6-aminopurin-9-ylmethyl)-5-chloro-3-(2-methoxy-phenyl)-3H-quinazolin-4-one;
2-(2-amino-9H-purin-6-ylsulfanylmethyl)-3-cyclopropyl-5-methyl-3H-quinazolin-4-one;
3-cyclopropylmethyl-5-methyl-2-(9H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3H-quinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2-(9H-purin-6-ylsulfanylmethyl)-3H-quinazolin-4-one;
2-(6-aminopurin-9-ylmethyl)-3-cyclohexyl-5-methyl-3H-quinazolin-4-one;
2-(2-amino-9H-purin-6-ylsulfanylmethyl)-3-cyclo-hexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)-dimethylamino-(9H-purin-6-ylsulfanylmethyl)-3H-quinazolin-4-one,
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydro-quinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylaminopurin-9-ylmethyl)-5-methyl-3-o-tolyl-3H-quinazolin-4-one;
5-methyl-2-(2-methyl-6-oxo-1,6-dihydro-purin-7-ylmethyl)-3-o-tolyl-3H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl)-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1,3,5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-diamino-pyrimidin-4-ylsulfanylmethyl)-5-methyl-3-o-tolyl-3H-quinazolin-4-one;
5-methyl-2-(5-methyl-[1,2,4]triazolo[1,5-a]pyrimidin-7-ylsulfanylmethyl)-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo[4,5-d]pyrimidin-3-yl-methyl)-5-methyl-3-o-tolyl-3H-quinazolin-4-one;
2-(7-amino-1,2,3-triazolo[4,5-d]pyrimidin-1-yl-methyl)-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-3 H-quinazolin-4-one;
2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one;
N-{2-[5-Methyl-4-oxo-2-(9H-purin-6-ylsulfanylmethyl)-4H-quinazolin-3-yl]-phenyl}-acetamide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid;
3-{2-[(2-dimethylaminoethyl)methylamino]phenyl}-5-methyl-2-(9H-purin-6-ylsulfanylmethyl)-3H-quin-azolin-4-one ;
3-(2-chlorophenyl)-5-methoxy-2-(9H-purin-6-ylsulfanylmethyl)-3H-quinazolin-4-one;
3-(2-chlorophenyl)-5-(2-morpholin-4-yl-ethylamino)-2-(9H-purin-6-ylsulfanylmethyl)-3H-quinazolin-4-one;
3-benzyl-5-methoxy-2-(9H-purin-6-ylsulfanymethyl)-3 H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[1-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2-(2-(1-methylpyrrolidin-2-yl)-ethoxy)-phenyl}-3H-quinazolin-4-one;
2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one;
2-{2-(1-(6-aminopurin-9-ylmethyl)-5-methyl-4-oxo-4H-quinazolin-3-yl]-phenoxy}-acetamide;
2-[(6-aminopurin-9-yl)methyl]-5-methyl-3-o-tolyl-3-hydroquinazolin-4-one;
3-(3,5-difluorophenyl)-5-methyl-2-[(purin-6-ylamino)methyl]-3-hydroquinazolin-4-one;
3-(2,6-dichlorophenyl)-5-methyl-2-[(purin-6-ylamino)methyl]-3-hydroquinazolin-4-one;
3-(2-Fluoro-phenyl)-2-[1-(2-fluoro-9H-purin-6-ylamino)-ethyl]-5-methyl-3-hydroquinazolin-4-one;
2-[1-(6-aminopurin-9-yl)ethyl]-3-(3,5-difluorophenyl)-5-methyl-3-hydroquinazolin-4-one;
2-[1-(7-Amino-[1,2,3]triazolo[4,5-d]pyrimidin-3-yl)-ethyl]-3-(3,5-difluoro-phenyl)-5-methyl-3H-quinazolin-4-one;
5-chloro-3-(3,5-difluoro-phenyl)-2-[1-(9H-purin-6-ylamino)-propyl]-3H-quinazolin-4-one;
3-phenyl-2-[1-(9H-purin-6-ylamino)-propyl]-3H-quinazolin-4-one;
5-fluoro-3-phenyl-2-[1-(9H-purin-6-ylamino)-propyl]-3H-quinazolin-4-one;
3-(2,6-difluoro-phenyl)-5-methyl-2-[1-(9H-purin-6-ylamino)-propyl]-3H-quinazolin-4-one;
6-fluoro-3-phenyl-2-[1-(9H-purin-6-ylamino)-ethyl]-3H-quinazolin-4-one;
3-(3,5-difluoro-phenyl)-5-methyl-2-[1-(9H-purin-6-ylamino)-ethyl]-3H-quinazolin-4-one;
5-fluoro-3-phenyl-2-[1-(9H-purin-6-ylamino)-ethyl]-3H-quinazolin-4-one;
3-(2,3-difluoro-phenyl)-5-methyl-2-[1-(9H-purin-6-ylamino)-ethyl]-3H-quinazolin-4-one;
5-methyl-3-phenyl-2-[1-(9H-purin-6-ylamino)-ethyl]-3H-quinazolin-4-one;
3-(3-chloro-phenyl)-5-methyl-2-[1-(9H-purin-6-ylamino)-ethyl]-3H-quinazolin-4-one;
5-methyl-3-phenyl-2-[(9H-purin-6-ylamino)-methyl]-3H-quinazolin-4-one;
2-[(2-amino-9H-purin-6-ylamino)-methyl]-3-(3,5-difluoro-phenyl )-5-methyl-3H-quinazolin-4-one;
3-{2-[(2-diethylamino-ethyl)-methyl-amino]-phenyl}-5-methyl-2-[(9H-purin-6-ylamino)-methyl]-3H-quinazolin-4-one;
5-chloro-3-(2-fluoro-phenyl)-2-[(9H-purin-6-ylamino)-methyl]-3H-quinazolin-4-one;
5-chloro-2-[(9H-purin-6-ylamino)-methyl]-3-o-tolyl-3H-quinazolin-4-one;
5-chloro-3-(2-chloro-phenyl)-2-[(9H-purin-6-ylamino)-methyl]-3H-quinazolin-4-one;
6-fluoro-3-(3-fluoro-phenyl)-2-[1-(9H-purin-6-ylamino)-ethyl]-3H-quinazolin-4-one;
2-[1-(2-amino-9H-purin-6-ylamino)-ethyl]-5-chloro-3-(3-fluoro-phenyl)-3H-quinazolin-4-one; and,
pharmaceutically acceptable salts and solvates thereof.

43. A method of inhibiting leukocyte transmigration, comprising:
selectively inhibiting phosphoinositide 3-kinase delta (PI3Kδ) activity in endothelial cells, thereby inhibiting leukocyte transmigration into inflamed tissue.

44. The method according to claim 43, wherein inhibiting comprises administering an amount of a phosphoinositide 3-kinase delta (PI3Kδ) selective inhibitor effective to inhibit leukocyte transmigration into inflamed tissue.

45. The method according to claim 43, wherein said inhibiting is in vitro.

46. The method according to claim 43, wherein said inhibiting is performed in an individual in need thereof.

47. The method according to claim 43, wherein the leukocyte transmigration is reduced by at least about twenty percent relative to leukocyte transmigration in endothelial cells wherein PI3Kδ activity has not been selectively inhibited.

48. The method according to claim 43, wherein the inflamed tissue is pulmonary tissue.

49. The method according to claim 46, wherein the individual has a condition selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), asthma, lupus nephritis, Crohn's disease, ulcerative colitis, necrotising enterocolitis, pancreatitis, neumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatoses, acute purulent meningitis, thermal injury, granulocyte transfusion associated syndromes, cytokine-induced toxicity, and spinal cord injury.

50. The method according to claim 44, wherein the PI3Kδ selective inhibitor is a compound having formula (I) or pharmaceutically acceptable salts and solvates thereof: 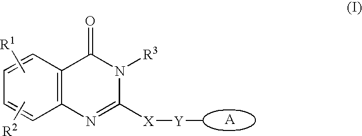
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO2N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a},C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a}is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a}groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

51. The method according to claim 44, wherein the PI3Kδ selective inhibitor is selected from the group consisting of:
2-(6-aminopurin-9-ylmethyl)-3-(2-chlorophenyl)-6,7-dimethoxy-3H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-5fur-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one;
5-chloro-3-(2-fluorophenyl)-2-(9H-purin-6-yl-sulfanylmethyl)-3H-quinazolin-4-one;
2-(6-aminopurin-9-ylmethyl)-5-chloro-3-(2-fluorophenyl)-3H-quinazolin-4-one;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophenyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl)-2-(9H-purin-6-yl-sulfanylmethyl)-3H-quinazolin-4-one;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl)-6,7-difluoro-2-(9H-purin-6-yl-sulfanylmethyl)-3H-quinazolin-4-one;
3-(2-chlorophenyl)-6-fluoro-2-(9H-purin-6-yl-sulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one;
2-(6-aminopurin-9-ylmethyl)-5-chloro-3-o-tolyl-3H-quinazolin-4-one;
2-(6-aminopurin-9-yl methyl )-5-chloro-3-(2-methoxy-phenyl)-3H-quinazolin-4-one;
2-(2-amino-9H-purin-6-ylsulfanylmethyl)-3-cyclopropyl-5-methyl-3H-quinazolin-4-one;
3-cyclopropylmethyl-5-methyl-2-(9H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3H-quinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2-(9H-purin-6-ylsulfanylmethyl)-3H-quinazolin-4-one;
2-(6-aminopurin-9-ylmethyl)-3-cyclohexyl-5-methyl-3H-quinazolin-4-one;
2-(2-amino-9H-purin-6-ylsulfanylmethyl)-3-cyclo-hexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)dimethylamino-(9H-purin-6-ylsulfanylmethyl)-3H-quinazolin-4-one;
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydro-quinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylaminopurin-9-yl methyl )-5-methyl-3-o-tolyl-3H-quinazolin-4-one;
5-methyl-2-(2-methyl-6-oxo-1,6-dihydro-purin-7-yl methyl )-3-o-tolyl-3 H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1 ,3,5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-diamino-pyrimidin-4-ylsulfanylmethyl)-5-methyl-3-o-tolyl-3H-quinazolin-4-one,
5-methyl-2-(5-methyl-[1,2,4]triazolo[1,5-a]pyrimidin-7-ylsulfanylmethyl)-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo[4,5-d]pyrimidin-3-yl-methyl)-5-methyl-3-o-tolyl-3H-quinazolin-4-one;
2-(7-amino-1,2,3-triazolo[4,5-d]pyrimidin-1-yl-methyl)-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-1-3H-quinazolin-4-one;
2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one;
N-{2-[5-Methyl-4-oxo-2-(9H-purin-6-ylsulfanylmethyl)-4H-quinazolin-3-yl]-phenyl}-acetamide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid;
3-{2-[(2-dimethylaminoethyl)methylamino]phenyl}-5-methyl-2-(9H-purin-6-ylsulfanylmethyl)-3H-quin-azolin-4-one;
3-(2-chlorophenyl)-5-methoxy-2-(9H-purin-6-ylsulfanylmethyl)-3H-quinazolin-4-one;
3-(2-chlorophenyl)-5-(2-morpholin-4-yl-ethylamino)-2-(9H-purin-6-ylsulfanylmethyl)-3H-quinazolin-4-one;
3-benzyl-5-methoxy-2-(9H-purin-6-ylsulfanylmethyl-3H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[1-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2-(2-(1-methylpyrrolidin-2-yl)-ethoxy)-phenyl}-3H-quinazolin-4-one;
2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one;
2-{2-( 1-(6-aminopurin-9-ylmethyl)-5-methyl-4-oxo-4H-quinazolin-3-yl]-phenoxy}-acetamide;
2-[(6-aminopurin-9-yl)methyl]-5-methyl-3-o-tolyl-3-hydroquinazolin-4-one;
3-(3,5-difluorophenyl)-5-methyl-2-[(purin-6-ylamino)methyl]-3-hydroquinazolin-4-one;
3-(2,6-dichlorophenyl)-5-methyl-2-[(purin-6-ylamino)methyl]-3-hydroquinazolin-4-one;
3-(2-Fluoro-phenyl)-2-[1-(2-fluoro-9H-purin-6-ylamino)-ethyl]-5-methyl-3-hydroquinazolin-4-one;
2-[1-(6-aminopurin-9-yl)ethyl]-3-(3,5-difluorophenyl)-5-methyl-3-hydroquinazolin-4-one;
2-[1-(7-Amino-[1,2,3]triazolo[4,5-d]pyrimidin-3-yl)-ethyl]-3-(3,5-difluoro-phenyl)-5-methyl-3H-quinazolin-4-one;
5-chloro-3-(3,5-difluoro-phenyl)-2-[1-(9H-purin-6-ylamino)-propyl]-3H-quinazolin-4-one;
3-phenyl-2-[1-(9H-purin-6-ylamino)-propyl]-3H-quinazolin-4-one;
5-fluoro-3-phenyl-2-[1-(9H-purin-6-ylamino)-propyl]-3H-quinazolin 4-one;
3-(2,6-difluoro-phenyl)-5-methyl-2-[1-(9H-purin-6-ylamino)-propyl]-3H-quinazolin-4-one;
6-fluoro-3-phenyl-2-[1-(9H-purin-6-ylamino)-ethyl]-3H-quinazolin-4-one;
3-(3,5-difluoro-phenyl)-5-methyl-2-[1-(9H-purin-6-ylamino)-ethyl]-3H-quinazolin-4-one;
5-fluoro-3-phenyl-2-[1-(9H-purin-6-ylamino)-ethyl]-3H-quinazolin-4-one;
3-(2,3-difluoro-phenyl)-5-methyl-2-[1-(9H-purin-6-ylamino)-ethyl]-3H-quinazolin-4-one;
5-methyl-3-phenyl-2-[1-(9H-purin-6-ylamino)-ethyl]-3H-quinazolin-4-one;
3-(3-chloro-phenyl)-5-methyl-2-[1-(9H-purin-6-ylamino)-ethyl]-3H-quinazolin-4-one;
5-methyl-3-phenyl-2-[(9H-purin-6-ylamino)-methyl]-3H-quinazolin-4-one;
2-[(2-amino-9H-purin-6-ylamino)-methyl]-3-(3,5-difluoro-phenyl)5-methyl-3H-quinazolin-4-one;
3-{2-[(2-diethylamino-ethyl)-methyl-amino]-phenyl}-5-methyl-2-[(9H-purin-6-ylamino)-methyl]-3H-quinazolin-4-one;
5-chloro-3-(2-fluoro-phenyl)-2-[(9H-purin-6-ylamino)-methyl]-3H-quinazolin-4-one;
5-chloro-2-[(9H-purin-6-ylamino)-methyl]-3-o-tolyl-3H-quinazolin-4-one;
5-chloro-3-(2-chloro-phenyl)-2-[(9H-purin-6-ylamino)-methyl]-3H-quinazolin-4-one;
6-fluoro-3-(3-fluoro-phenyl)-2-[1-(9H-purin-6-ylamino)-ethyl]-3H-quinazolin-4-one;
2-[1-(2-amino-9H-purin-6-ylamino)-ethyl]-5-chloro-3-(3-fluoro-phenyl)-3H-quinazolin-4-one; and,
pharmaceutically acceptable salts and solvates thereof.
